(19) United States
(12) Patent Application Publication
(10) Pub. No.: US 2004/0002063 A1
(43) Pub. Date: Jan. 1, 2004

(54) Rapid vaccinia antibody detection device, method and test kit

(75) Inventors: Hermes K.W. Chan, Halifax (CA); Spencer Lee, Halifax (CA)

Correspondence Address: EMCH, SCHAFFER, SCHAUB & PORCELLO CO, P O BOX 916, ONE SEAGATE SUITE 1980, TOLEDO, OH, 43697

(73) Assignee: MedMira Inc., Toronto, ON (CA)

(21) Appl. No.: 10/440,031

(22) Filed: May 16, 2003

Related U.S. Application Data
(60) Provisional application No. 60/378,063, filed on May 16, 2002.

(51) Int. Cl. C12Q 1/70; C12M 1/34

(52) U.S. Cl. 435/5; 435/287.2

(57) ABSTRACT

The invention relates to a rapid vaccinia antibody detection device, method and test kit for the detection of vaccinia antibody in a serum, plasma or whole blood test sample utilizing a purified vaccinia cell lysate as the capture antigen. The detection device operates on the basis of a 2-step flow-through format in use with a push buffer.

### FIELD OF INVENTION

The invention relates to a rapid vaccinia antibody detection device, method and test kit for the detection of vaccinia antibody in a serum, plasma or whole blood test sample utilizing a purified vaccinia cell lysate as the capture antigen. The detection device operates on the basis of a 2-step flow-through format in use with a push buffer.

### BACKGROUND OF INVENTION

The global fear of biological warfare has spurred the medical community to implement extra precautionary measures. As a biological weapon, the smallpox virus would pose a serious threat due to its case-fatality rate of 30% or more in unvacinnated persons and to the current unavailability of treatment (Henderson et al., 1999). The waning or non-existent human immunity to the smallpox virus is a great concern in consideration of the fact that the virus has developed a knack for rapid growth in humans, effective spread and persistence.

Smallpox may have been used as a biological weapon in North America as early as the mid- to late 1700's when British soldiers initiated outbreaks in Native Americans by distributing blankets that had been used by smallpox patients. Affected tribes lost more than 50% of their population (Henderson et al., 1999) due to the fact that most humans are subject to the ease of transmission and high rate of infectivity of this disease. With Edward Jenner's demonstration of the role of the vaccinia virus' protective abilities against smallpox in 1796, (Henderson et al., 1999; Kaufman et al., 2001), its potential threat of a bioweapon was greatly diminished. The World Health Organization's smallpox eradication campaign, initiated in 1967, saw the last naturally occurring case in the world in Somalia in 1977 and brought the end to mass vaccinia vaccination programs.

The vaccinia virus, a live, animal poxvirus, was a highly effective vaccine responsible for the successful eradication of smallpox over 30 years ago (Kaufman et al., 2001). World-recognized, the vaccinia virus has been safely administered to persons of all ages, providing humans with protective antibodies against smallpox. Although with today's threats, a preventative mass vaccination program will not be possible until increased amounts of vaccine are made available. A recent press release by the United States federal government indicates their decision to select pharmaceutical companies to manufacture and stockpile millions of doses of smallpox vaccine in order to protect the American people (Kaufman et al., 2001; Rosenthal et al., 2001). However, the decision to undertake such a massive vaccination program must be weighed against the risk of vaccination complications.

Routine vaccination against smallpox ceased in the United States approximately 30 years ago, leaving today's population highly susceptible to reintroduction of this devastating disease. Statistics show that approximately 114 million people in the United States (42%) are aged 29 years or younger and therefore have not been vaccinated against this disease (Henderson et al., 1999). Furthermore, the immune status of those people who were vaccinated prior to this time is not clear (Rosenthal et al., 2001). The duration of immunity has never been satisfactorily measured but it appears, in general, that a substantial protective antibody decline has been identified during the five to ten year period following smallpox vaccination (Henderson et al., 1999). The underlying strategy in immunizing humans against a particular disease is to prepare an innocuous form of the infectious organism which retains the antigen responsible for establishing protective immunity.

The vaccinia virus vaccine is a live virus from the Poxvirus family which produces defences to smallpox infection in humans. Vaccinia, a eukaryotic virus, reproduces entirely within the cytoplasm of a host cell. It is a lytic virus, i.e. a virus, the replication of which in a cell results in lysis of the cell. The virus is considered non-oncogenic. The virus has been used for approximately 200 years in vaccines for inoculation against smallpox and the medical profession is well acquainted with the properties of the virus when used in a vaccine. Although inoculation with vaccinia is not without risk, the risks are on the whole well known and well defined and the virus is considered relatively benign.

The traditional method for determining success of vaccination against smallpox was simply a qualitative observation of the lesion or scar by a health care professional. Obviously, this method of evaluation is very subjective and requires a vast amount of experience and observation to obtain valid results. A serum neutralization test, process by which serial dilutions of a patient's serum are tested for the presence of antibodies to the vaccinia virus, can be performed to test the ability of the patient sample to neutralize the infectivity of the virus. This technique, however, is very involved and time consuming, taking days to obtain results. On this basis, a more simplistic and rapid approach to the detection of antibodies to vaccinia virus without the aid of complicated instruments and the requisite skills and knowledge of professionally trained personnel would be highly desirable. This is especially the case in the event of possible biochemical warfare wherein an emergency response program must be implemented quickly and efficiently to control and prevent possible contamination of the highly infectious disease within a population.

Accordingly, the present invention provides a rapid vaccinia antibody detection device, method and test kit for the detection of vaccinia antibody in a serum, plasma or whole blood test sample utilizing a purified vaccinia cell lysate as the capture antigen. The detection device, method and push buffer are efficient, reliable and practical to perform. Moreover, the simple design of rapid vaccinia detection device is less costly to manufacture and perform compared to other conventional systems, thus making it more economically feasible and affordable to use. The present invention will be able to provide health professionals a rapid way to monitor the success of the smallpox vaccination process.

### SUMMARY OF THE INVENTION

The rapid vaccinia detection device operates on the basis of a simple 2-step flow-through format in use with a push buffer. The detection device is a dual component flow-through system comprising a test unit in combination with a detachable post-filter unit which are capable of receiving a serum, plasma or whole blood sample and push buffer, respectively. The push buffer serves as a combination washing, diluting, wetting and resolubilizing reagent, without sacrificing the sensitivity or specificity of the test. Additionally, the buffer is formulated to preserve and optimize protein stability, as well as minimize, if not eliminate, non-specific interactions that might lead to the generation of a false signal.

According to the present invention there is provided a rapid vaccinia antibody detection device, method and test kit for the detection of vaccinia antibody in a serum, plasma or whole blood test sample which utilizes a purified vaccinia cell lysate as the capture antigen. The rapid detection device and method are useful in the detection of protective antibodies for smallpox, which are produced in the human body in response to vaccinia virus vaccination. The rapid vaccinia antibody detection test is designed to be used as a follow-up to vaccination for determination of the success of vaccination, or as a screen test for individuals previously vaccinated during the smallpox eradication program to aid in determination of the requirement for revaccination. Accordingly, the user will be able to determine the success of an individual vaccination, or may be able to indicate the presence or absence of protective antibodies in the serum, plasma or whole blood test sample of an individual vaccinated during the smallpox eradication program 30 or more years ago.

Using vaccinia viral lysate as a capture antigen, the rapid vaccinia antibody detection test uses flow-through technology consisting of an immunoreactive nitrocellulose membrane, a push buffer and dehydrated gold conjugate cap. A drop of specimen can simply be added to the reactive membrane and the immunocomplex visualized by the addition of a proprietary colloidal gold conjugate colorimetric detection agent.

One of the advantages of the rapid vaccinia antibody detection device and method of the present invention is that it is designed to provide a quick portable, safe and cost-effective method for antibody testing by providing fast and accurate results. It is completely self-contained, requiring no refrigeration for storage or transport and no special equipment other than a standard laboratory centrifuge if a serum/plasma specimen is used. All the reagents and materials required to perform the detection test are provided in a single ready-to-use format. The push buffer provided with the device is a multi-functional reagent which eliminates the potential error for mix-up of reagents during the testing procedure which can lead to erroneous results.

Another advantage of the rapid vaccinia antibody detection device and method is that it provides a qualitative, in vitro diagnostic test for the detection of antibodies to the vaccinia virus in human serum, plasma, or whole blood. Accordingly, the rapid vaccinia antibody detection device and method of the present invention is highly effective in qualifying the success of vaccinia vaccination.

The rapid vaccinia antibody detection test utilizes a vaccinia viral lysate coated onto a membrane matrix to capture the antibodies to vaccinia present in human serum, plasma or whole blood when the content of the specimen is placed on the immunoreactive membrane. The captured vaccinia antibodies are visualized through a colorimetric reaction with a colloidal gold-protein A conjugate as the preferred indicator reagent. When the conjugate binds to the vaccinia antibodies, a distinctive horizontal red line appears on the test membrane. A positive test result is obtained when both the red horizontal test line and a red vertical procedural control line are visible at the end of the test procedure. In contrast, a negative result due to the absence of anti-vaccinia virus antibodies is indicated by the presence of only the vertical red procedural control line on the test membrane. If there is no vertical procedural control line present, the test result is invalid.

Using the simplified device and single buffer reagent of the present invention, a qualitative detection test can be performed and read easily, requires a minimum number of steps, does not require lengthy incubation periods, and is highly sensitive, specific and reliable.

Typically, as little as a single drop (50 mL) of a fluid sample is needed to perform the detection test. Moreover, the resulting device and methodology is particularly advantageous in that it is not only convenient and simple to use, but the device and reagents can be stored at room temperature for long periods of time without diminishing the activity or sensitivity of the detection test.

The kinetics of the reaction between the target analyte, i.e. anti-vaccinia virus antibodies, and the indicator reagent are extremely rapid and complete because the detection device and procedure operates on the basis of a flow-through format. Moreover, the method of the present invention improves the accuracy of the detection test compared to conventional assays since the final step of the detection test involves the addition of resolubilized indicator reagent to the test sample after it has previously complexed with the capture reagent, i.e. purified vaccinia viral lysate.

The test unit comprises (1) a reaction zone containing a purified vaccinia viral lysate as the capture reagent that can specifically recognize and bind to antibodies against vaccinia virus, (2) an absorbent zone supporting the reaction zone, and (3) optionally, a blood separation zone contiguous with the reaction zone. The reaction zone of the test unit is oriented so that the label zone of the post-filter unit can be brought into fluid communication therewith after the fluid test sample is applied to the test unit. In a preferred embodiment, the reaction zone is comprised of a porous membrane compatible for immobilization of the capture reagent and has low non-specific binding for the indicator reagent. Any non-specific binding sites on the surface of the porous reaction membrane are inactivated by applying a protein blocking agent. The specificity and affinity of the vaccinia viral lysate immobilized on the reaction membrane allows it to efficiently bind and concentrate any anti-vaccinia virus antibodies contained in a fluid sample within a defined region as the test sample diffuses by capillary action from the reaction membrane to the absorbent zone directly underneath.

To facilitate the detection of anti-vaccinia virus antibodies in a whole blood sample, an alternate embodiment of the present invention provides a test unit capable of receiving and separating the fluid portion of a whole blood sample from the red blood cells (RBC), while transporting the RBC-free fluid portion of the sample to the reaction zone for the detection of the analyte. This particular feature is useful in preventing any interference during visualization of a colour reaction for the detection of anti-vaccinia virus antibodies (i.e. the use of “direct” labels which provide a visually detectable signal without the aid of instruments) and also avoids the necessity to obtain a preliminary extraction of serum or plasma in settings where proper equipment to perform such a procedure is unavailable.

Thus, in the case where the fluid sample to be analyzed is a whole blood sample, the test unit optionally features a separate blood separation zone contiguous with the reaction zone. In general, the blood separation zone functions to selectively retain cellular components (i.e. red blood cells) contained within the whole blood sample and deliver the remaining components of the blood sample, including any anti-vaccinia virus antibodies, to the reaction zone. A first end of the blood separation zone, located a short lateral distance from the reaction zone, defines a region for receiving a whole blood sample during the initial stage of the detection test. A second end of the blood separation zone is contiguous with, and thus in direct fluid communication with, the reaction zone thereby promoting the capillary movement of the RBC-free fluid portion of the blood sample from the application zone to the reaction zone for direct detection of the presence of any anti-vaccinia virus antibody. Thus, in effect, the blood separation material functions as a lateral flow material for the selective removal of an effective amount of red blood cells from the whole blood sample to prevent interference with the visual detection of the analyte, while allowing other components of the sample to flow with relatively unimpaired movement through the test unit.

In a preferred embodiment, the blood separation zone is an elongate or rectangular strip of porous material employing a hydrophobic carrier or backing and having intrinsic properties which enable it to preferentially entrap or retain the red blood cells in the sample within the blood separation zone. The carrier or backing provides support for the blood separation material and reduces seepage of the whole blood sample as the RBC-free fluid portion migrates along the material towards the reaction zone.

The second component of the device, namely the post-filter unit, comprises a label zone permeated with a dried indicator reagent and which is capable of being placed in transient fluid communication with the reaction zone of the test unit shortly following application of the test sample to the test unit. Impregnating the label zone of the post-filter unit with a permanently detectable indicator reagent eliminates the need to perform separate resolubilization steps involving precise measuring, adding and premixing with a suitable solvent, thereby increasing the possibility of user error. In a preferred embodiment, the label zone comprises a filter medium selected on the basis of having a pore size large enough so that when the dried indicator reagent is resolubilized by addition of the push buffer, it will easily flow through an exposed area of the porous filter medium by the process of diffusion. The shape and dimensions of the post-filter unit are such that it will hold and effectively channel the push buffer through the porous filter medium when the label zone is placed in transient fluid communication with the reaction zone of the test unit during the test procedure.

According to another important aspect of the invention, methods and devices are provided utilizing “direct” labeled specific binding materials (i.e. colloidal particle labeled materials) which are dried onto a filter medium and hence, are capable of being rapidly resolubilized and transported to the reaction zone in the presence of the push buffer. Direct labels are well known in the art and highly advantageous for their use in rapid diagnostic systems. Direct labels are capable of producing a visually detectable signal without the aid of instrumentation or the addition of ancillary reagents and are stable when stored in the dry state. Supplying the indicator reagent by way of incorporating it within the filter medium in a dried form provides an inexpensive and convenient means of storing such reagent. The preferred label for carrying out diagnostic assays is colloidal metal particles, more preferably colloidal gold, although other direct labels may be employed which include, but are not limited to, non-metal sols, dye sols, latex particles, carbon sol, and liposome contained colored bodies.

According to a further important aspect of the present invention, there is provided a push buffer in the detection test which does not require ancillary additives, or the maintenance and inspection by laboratory instruments. More importantly, however, is the multifunctional nature of the push buffer which enables it to serve as a combination wash solution, diluent, resolubilization and solvent transport reagent, thereby eliminating the need for several separate solutions and steps to be performed during the detection test protocol. The multifunctional nature of the push buffer greatly simplifies the detection test procedure by reducing the time and manual steps required to perform the test, thereby minimizing the likelihood for user error. In addition, utilizing the push buffer in a flow-through format promotes quick release and enhanced mass transfer of the dried indicator reagent from the post-filter unit to the test unit immediately following resolubilization. Other functional properties exhibited by the push buffer are that it maintains protein stability, thereby preserving and optimizing the specific binding reaction that occurs between complementary binding members, i.e. capture reagent and target analyte. Moreover, upon resolubilization of the dried indicator reagent, the push buffer helps to optimize signal generation in the case of a specific binding reaction and minimize nonspecific binding to the reaction membrane that might otherwise lead to the generation of a false signal.

According to yet a further aspect of the present invention, there is provided a simple 2-step procedure for performing the rapid vaccinia antibody detection method comprising (1) depositing a fluid test sample onto the reaction zone of the test unit, or if a whole blood sample, onto a first end of a blood separation zone, shortly thereafter bringing the test unit and the post-filter unit into operable association such that the label zone of the post-filter unit is in transient fluid communication with the reaction zone of the test unit, and (2) adding the push buffer to the post-filter unit followed by removal of the post-filter unit to observe the test result. Following addition of the push buffer to the post-filter unit, the buffer diffuses through the label zone to reconstitute the indicator reagent and transport it to the reaction zone where it will bind with any captured anti-vaccinia virus antibody. If anti-vaccinia virus antibody is present in the fluid sample, a detectable signal will appear in the reaction zone which can be visually inspected for color and thus, a determination of the presence or absence of anti-vaccinia virus antibody made following removal of the post-filter unit.

The present invention also provides a rapid vaccinia antibody detection test kit for use in the determining whether there is any anti-vaccinia antibody in a fluid test sample. Essentially, the kit comprises in a packaged combination: the rapid vaccinia antibody detection device comprising both the test unit and post-filter unit as described above; a push buffer for reconstitution of the dried indicator reagent; and instructions for performing the detection test. The test kit preferably includes a suitable container for housing the test unit and the post-filter unit in order to safeguard the solid phase materials and dried indicator reagent from contamination, as well as to provide ease and convenience in handling of the detection test device. Optionally, the test kit also includes a means for applying the test sample and push buffer to the test unit and post-filter unit, respectively (e.g. disposable pipettes).

### BRIEF DESCRIPTION OF THE DRAWINGS

FIG. 1A is a diagrammatic illustration of a test sample applied to the porous reaction membrane of the test unit which contains anti-vaccinia virus antibody;

FIG. 1B is a diagrammatic illustration of the anti-vaccinia virus antibody complexed with vaccinia viral lysate as the capture reagent after the test sample has completely diffused through the reaction membrane and into the absorbent material of the test unit;

FIG. 1C is a diagrammatic illustration of the post-filter unit in fluid communication with the reaction membrane of the test unit, to which the push buffer is added;

FIG. 1D is a diagrammatic illustration of resolubilized indicator reagent reacted with complexed vaccinia viral lysate and anti-vaccinia virus antibody following addition of the push buffer to the post-filter unit;

FIG. 2A is a diagrammatic illustration of a test sample applied to the porous reaction membrane of the test unit which does not contain anti-vaccinia virus antibody;

FIG. 2B is a diagrammatic illustration of uncomplexed vaccinia viral lysate as the capture reagent after the test sample has diffused through the reaction membrane and into the absorbent material of the test unit;

FIG. 2C is a diagrammatic illustration of the post-filter unit in fluid communication with the reaction membrane of the test unit, to which the push buffer is added;

FIG. 2D is a diagrammatic illustration of unreacted indicator reagent following resolubilization by the push buffer after diffusing through the reaction membrane and into the absorbent material of the test unit;

FIG. 3 shows an exploded cross-sectional view of a preferred embodiment of a suitable container which houses the test unit and the post-filter unit; and

FIG. 4 shows an enlarged cross-sectional view of the container of FIG. 4 in its assembled form;

FIG. 5 is a diagrammatic illustration of a second embodiment of a portion of the test unit comprising a material defining the blood separation zone in fluid communication with the reaction zone; and

FIGS. 6A and 6B are a diagrammatic illustration of top plan views of the top and bottom members of a 2-reservoir test cartridge for receiving and analyzing a whole blood sample.

While this invention is satisfied by embodiments in many different forms, there will herein be described in detail preferred embodiments of the invention, with the understanding that the present disclosure is to be considered as exemplary of the principles of the invention and is not intended to limit the invention to the embodiments illustrated and described. The scope of the invention will be measured by the appended claims and their equivalents.

### DETAILED DESCRIPTION

Unless defined otherwise, all technical and scientific terms used herein have the same meaning as commonly understood by one of ordinary skill in the art to which this invention belongs. As employed throughout the disclosure, the following terms, unless otherwise indicated, shall be understood to have the following meanings.

It must also be noted that, as used in the specification, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

Capilliary Action—as used herein, the term “capillary” includes a capillary or other channel or pathway which permits a liquid to traverse the absorbent material of the invention. The absorbent material is in capillary communication with the reaction membrane of the test unit and is selected to have a capillary pore size so as to induce flow of liquid through the reaction membrane without the use of external means when the hydrostatic pressure of the sample and subsequent addends used in the detection test are not sufficient to induce flow through the reaction membrane. The absorbent material may also provide support for the reaction membrane.

Fluid Sample—the fluid sample is tested to form a detectible reaction product on the reaction membrane of the test unit. In preferred embodiments of the detection test, the fluid sample is biologically derived (e.g. whole blood, plasma, serum, urine, saliva, etc.) and is suspected to include as the target analyte, anti-vaccinia virus antibody capable of being bound by the vaccinia viral lystate immobilized on the reaction membrane as the capture reagent.

Indicator Reagent—a conjugate comprised of a specific binding member to the anti-vaccinia virus antibody and a label that is capable of being visually detected. A preferred indicator reagent of the present invention is protein A labeled with colloidal gold. Other indicator reagents for anti-vaccinia virus antibody as the target analyte would include, for example, goat antihuman IgG labeled with colloidal gold.

Label—a label may be any molecule bound or conjugated to a specific binding member which can produce a signal. In the subject invention, the label is preferably a “direct” label which is capable of spontaneously producing a detectible signal without the addition of ancillary reagents and will be easily detected by visual means without the aid of instruments. The preferred embodiment of the invention uses colloidal gold particles as the label. Other suitable labels may include other types of colloidal metal particles, minute colored particles, such as dye sols, and coloured latex particles. Many such substances will be well known to those skilled in the art.

Solid Phase—methods have also been developed for carrying out “solid phase” assays wherein immunological reactions are carried out in solution on solid substrates including those which are porous or fibrous materials. According to such procedures, porous carrier materials are fashioned into strips or other forms to which the capture reagent, e.g. antibodies, antigens) are immobilized by adsorption, absorption or covalent bonding.

In a preferred embodiment of the invention, the type of method employed is based on an immunofiltration dotting assay system for the purposes of convenience and utility wherein components of a vaccinia viral lysate have been immobilized onto a solid support to effect a reaction. Immunofiltration dotting techniques are well known in the art.

1.0 Introduction

The detection device useful in the practice of the invention is a dual component flow-through system comprising a test unit and a post-filter unit capable of receiving the fluid sample and push buffer, respectively. The test unit comprises a reaction zone containing purified vaccinia viral lysate that can specifically bind with anti-vaccinia virus antibody, an absorbent zone supporting the reaction zone, and optionally, a blood separation zone contiguous with the reaction zone. The reaction zone of the test unit is oriented so that the label zone of the post-filter unit can be brought into transient fluid communication therewith shortly after the fluid test sample is applied to the reaction zone of the test unit. To facilitate the detection of anti-vaccinia virus antibody in a whole blood sample, an alternate embodiment of the present invention provides a test unit further comprising a blood separation zone contiguous with the reaction zone, whereby a first end of the blood separation zone located a short lateral distance from the reaction zone defines a region for receiving the whole blood sample. A second end of the blood separation zone slightly overlaps with the reaction zone so as to be in direct fluid communication therewith. The post-filter unit comprises a label zone containing a dried indicator reagent and is capable of being placed in transient fluid communication with the reaction zone of the test unit during the detection test procedure.

The detection test protocol is a simple 2-step procedure involving (1) depositing a fluid test sample onto the reaction zone of the test unit, or if a whole blood sample, onto a first end of the blood separation zone, shortly thereafter bringing the test unit and the post-filter unit into operable association such that the label zone of the post-filter unit is in transient fluid communication with the reaction zone of the test unit, and (2) adding the push buffer to the post-filter unit and removing the post-filter unit to observe the test result. The push buffer passively diffuses through the label zone of the post-filter unit to resolubilize the indicator reagent and transport it to the reaction zone of the test unit where it will bind to anti-vaccinia virus antibody that has complexed with the vaccinia viral lysate. If anti-vaccinia virus antibody is present in the fluid sample, a detectable signal will appear in the reaction zone which can be easily visualized following removal of the post-filter unit from the test unit. An advantage provided by the methodology of the present invention is the enhanced sensitivity and reliability of the test. This is achieved by maximizing the opportunity for thorough capture of the analyte, even at low concentrations, and avoiding the implementation of method steps which increase the likelihood of contamination of the sample and reagents.

2.0 Sandwich Technique

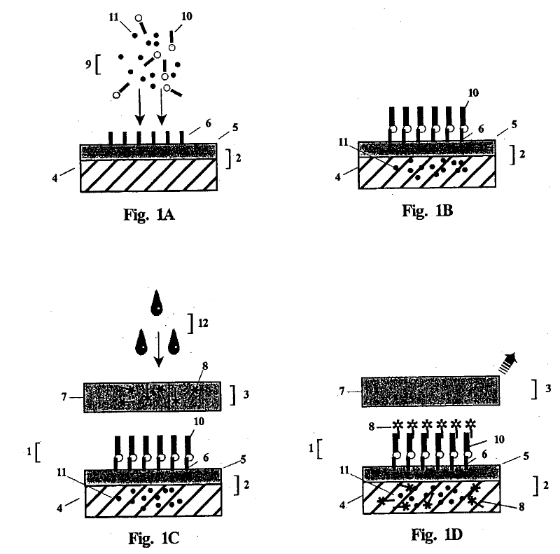

FIG. 1 is a diagrammatical illustration showing the method of the invention using the rapid vaccinia antibody detection device. In this particular instance, FIG. 1A shows a fluid test sample 9 containing anti-vaccinia virus antibody 10, as well as other non-essential components 11, which is applied to the reaction zone 5 of the test unit 2. As the fluid sample 9 diffuses through the reaction zone 5 and into the absorbent zone 4 underneath, the free anti-vaccinia virus antibody 10 comes into contact with components of the vaccinia viral lysate 6 and forms a complex, while unbound non-essential component 11 continues to be drawn into the absorbent zone 4 below (FIG. 1B). As shown in FIG. 1C, the label zone 7 of the post-filter unit 3 is subsequently brought into fluid communication with the reaction zone 5 of the test unit 2 prior to the addition of the push buffer 12. Immediately following resolubilization of the dried indicator reagent 8 by the buffer 12, the indicator reagent 8 is transported to the reaction zone 5 of the test unit 2, where it will bind with any anti-vaccinia antibody 10 that has complexed with components of the vaccinia viral lysate 6. The binding reaction of the indicator reagent 8 with anti-vaccinia virus antibody 10 produces a visually detectable signal thereby indicating a positive reaction that is easily observed following removal of the post-filter unit 3, as per FIG. 1D.

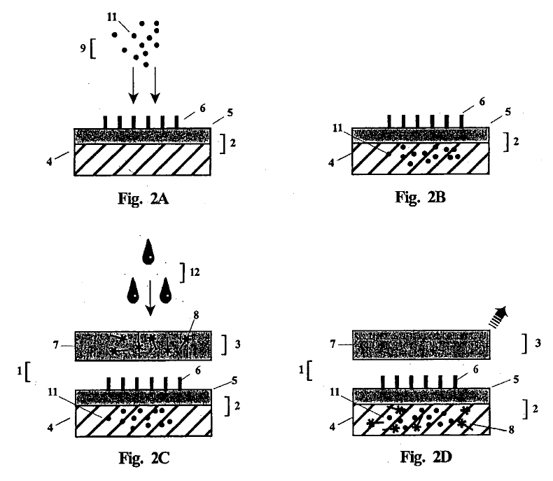

FIG. 2A is a diagrammatical illustration showing the method of the invention using the rapid vaccinia antibody device when a fluid test sample 9 devoid of anti-vaccinia virus antibody is applied to the reaction zone 5 of the test unit 2. As the fluid sample 9 diffuses through the reaction zone 5 and into the absorbent zone 4 underneath, the non-essential components 11 completely bypass the components of the vaccinia viral lysate 6 (FIG. 2B). As shown in FIG. 2C, the label zone 7 of the post-filter unit 3 is subsequently brought into fluid communication with the reaction zone 5 of the test unit 2 prior to the addition of the push buffer 12. Immediately following resolubilization of the dried indicator reagent 8 by the buffer 12, the indicator reagent 8 is transported to the test unit 2, where it diffuses through the reaction zone 5, pass the components of the vaccinia viral lysate 6 and into the absorbent zone 4 below due to the absence of any anti-vaccinia virus antibody complexed with the lysate 6. Following removal of the post-filter unit 3, a color signal will not be detected thereby indicating a negative result due to the absence of binding between the indicator reagent 8 and complexed anti-vaccinia virus antibody.

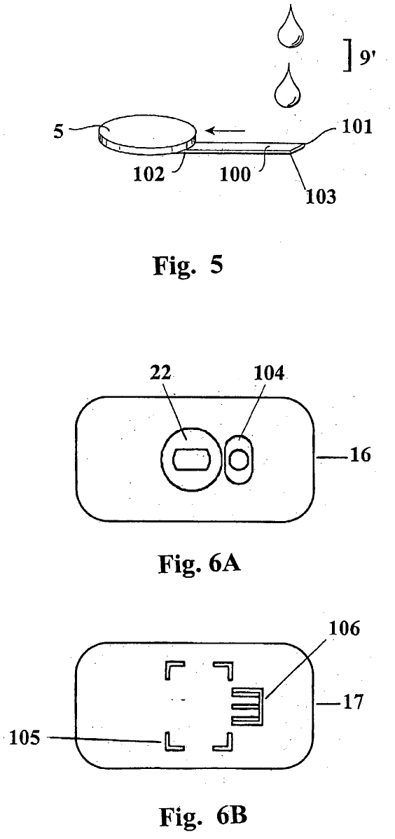

To facilitate the detection of a target analyte in a whole blood sample, an alternate embodiment of the present invention provides a test unit having a blood separation zone capable of receiving and separating the fluid portion of a whole blood sample from the red blood cells (RBC), while transporting the RBC-free fluid portion, including any analyte, to the reaction zone for direct analysis. As shown in FIG. 5, the blood separation zone 100 is preferably an elongate or rectangular strip of porous material employing a hydrophobic carrier 103. The preferred material for the blood separation zone 100 is selected on the basis of having intrinsic properties which enable it to preferentially entrap or retain the red blood cells in the sample 9′ as the fluid portion migrates in a lateral direction towards the reaction zone 5. Although the shape and dimensions are not critical, preferably the blood separation zone 100 is a rectangular form having dimensions suitable for allowing efficient removal of a substantial amount of red blood cells from the whole blood sample 9′ prior to the RBC-free fluid portion of the sample 9′ arriving at the reaction zone 5. Thus, in effect, the blood separation material functions as a lateral flow material for the selective removal of an effective amount of red blood cells from the whole blood sample 9′ so as to avoid interference with the visual detection of the analyte, while allowing other components of the sample, including any analyte, to flow with relatively unimpaired movement to the reaction zone 5.

A first end 101 of the blood separation zone 100, located a short lateral distance from the reaction zone 5, defines a region for receiving the whole blood sample 9′ during the initial stage of the detection test protocol. A second end 102 of the blood separation zone 100 is contiguous with and overlaps slightly with the reaction zone 5, so as to be in direct fluid communication with the reaction zone 5, thereby promoting the capillary movement of the RBC-free fluid portion of the blood sample 9′ from the first end of the blood separation zone 100 to the reaction zone 5. The blood separation zone 100 and the reaction zone 5 must contact one another in order to ensure optimal transfer of the sample from one zone to the other. Therefore, it is preferably that the blood separation zone 100 and the reaction zone 5 overlap with one another slightly as opposed to being abutted to one another.

The hydrophobic carrier 103 affixed to the lower surface of the blood separation zone 100 provides support and reduces seepage of the fluid phase while the RBC-free fluid portion of the whole blood sample migrates towards the reaction zone 5. Suitable materials for use as a carrier 103 include, for example, polycarbonate, polyethylene, Mylar, polypropylene, vinyl, cellophane and polystyrene, etc. as well as water-proofed or water-resistant cardboard or similar materials The carrier 103 may be affixed either directly or indirectly to the blood separation material by means of an adhesive. The carrier 103 is preferably similar in shape and size to the blood separation zone 100.

Thus, the two-step method protocol optionally employs a simultaneous separation of red blood cells from a whole blood sample 9′ in order to permit testing for anti-vaccinia virus antibody without the requirement for additional steps. For example, in the case where a whole blood sample 9′ contains anti-vaccinia virus antibody, the sample 9′ is simply applied to the first end of the blood separation zone 100 of the test unit 2, rather than the reaction zone 5. As the RBC-free fluid portion of the blood sample 9′ migrates in a lateral direction to arrive at the reaction zone 5, the free anti-vaccinia virus antibody 10 eventually comes into contact with components of the vaccinia viral lysate 6 and forms a complex. Thus, similar to the method step shown in FIG. 2B, unbound non-essential components 11 are drawn into the absorbent zone 4 located beneath the reaction zone 5. The label zone 7 of the post-filter unit 3 is subsequently brought into fluid communication with the reaction zone 5 of the test unit 2 prior to the addition of the push buffer 12 (refer to FIG. 1C). Immediately following resolubilization of the dried indicator reagent 8 by the buffer 12, the indicator reagent 8 is transported to the reaction zone 5 of the test unit 2, where it will bind with any anti-vaccinia virus antibody 10 that has complexed with components of the vaccinia viral lysate 6. The binding reaction of the indicator reagent 8 with the anti-vaccinia virus antibody 10 produces a visually detectable signal thereby indicating a positive reaction that is easily observed following removal of the post-filter unit 3, as per FIG. 1D.

3.0 Test Unit

As described above, the diagnostic device of the present invention comprises, as a first component, a test unit having a reaction zone containing immobilized components of a preferably purified vaccinia viral lysate that can specifically bind to anti-vaccinia virus antibody, an absorbent zone supporting the reaction zone, and optionally, a blood separation zone contiguous with the reaction zone. The reaction zone of the test unit is oriented so that the label zone of the post-filter unit can be brought into transient fluid communication therewith shortly after the fluid test sample is applied to the test unit.

3.1 Reaction Zone

The selection of the material for the reaction zone is not critical to the invention. The materials used to fabricate the device of the present invention are well known in the art. Porous or fibrous materials, such as those described in U.S. Pat. Nos. 4,670,381, 4,632,901, 4,666,863, 4,459,361, 4,517,288, and 4,552,839, may be composed singly or in combination of glass fibers, cellulose acetates, nylon, or various synthetic or natural materials.

The preferred material of the reaction zone is a membrane which has a pore size permitting separation and filtration of other non-essential components from the fluid biological sample being tested. The criteria of selection is that the membrane has controlled pore sizes preferably ranging from 0.05 to 20.0 microns, more preferably ranging from 0.2 to 0.8 microns. The flow of the aqueous reagents can be controlled through diffusion, filtration, positive or negative pressure, and the membrane should have low nonspecific binding for the indicator reagent before or after treatment with reagents such as proteins, detergents, or salts. There are many porous membrane, films, or papers available commercially which have controlled hydrophobicity and are suitable for the practice of the invention. The porous reaction membrane can be any shape and thickness but usually is flat and thin. The absorption, diffusion or filtration of the liquid phase of the reactants from the solid phase particles in the separation step of the detection test can be facilitated by the addition of a fibrous or hydrophilic material (absorbent pad) in contact with the underside of the porous reaction membrane. The size of the area exposed to the solid phase particles can be controlled by using a device comprised of a hydrophobic material such as plastic, plastic laminate, or other similar substance that is placed in contact with the porous film and seals the porous reaction membrane such that only a surface area no greater than 150 mm² is exposed to the particulate solid phase.

Another factor to be considered is that the material of the porous reaction membrane be selected on the basis that it is compatible for immobilization of the vaccinia viral lysate. The reaction membrane may be any suitable porous material so long as the performance of the detection test is not adversely affected. Suitable materials include nitrocellulose (supported or unsupported), glass fiber, polyester, cellulose nitrate, polyester, polycarbon, nylon, and other natural and synthetic materials which can be coupled directly or indirectly to components of the vaccinia viral lysate. Some of these materials may comprise negative charges to assist in immobilization, such as cellulose nitrate which has partial negative charges contributed by the nitro groups.

In some cases commercial filters are available that have immobilized to their internal and/or external surfaces a reactant for the attachment of biological molecules, such as antibodies or antigens, to the surfaces. Examples of various filters include cellulosic filters (filter papers), polyamide membranes (e.g. numerous variations of polyamide membranes are manufactured by the Pall Corporation), and various other microporous membranes, such as those available commercially from Amicon, Geleman, and Schleicher & Schuell. For example, the following membranes are available from Pall Corporation: Biodyne®, a N66 polyamide microporous membrane (U.S. Pat. No. 4,340,479 issued to Pall); Carboxydyne®, a hydrophilic, microporous, skinless nylon 66 membrane with control surface properties characterized by carboxyl functional groups at Its surfaces; and Immunodyne™, a modified Carboxydyne® membrane prepared by treating. a Carboxydyne® membrane with trichloro-s-triazine. Other microporous membranes, prepared by the Millipore Corporation, are described in U.S. Pat. Nos. 4,066,512 and 4,246,339.

Other materials may be pre-treated to provide a charged membrane. For example, polyester can be derivatized with carboxyl or amino groups to provide either a negatively or positively charged membrane. Nylon can be treated with acid to break peptide bonds to provide positive charges (from the amine groups) and negative charges (from the carboxyl groups).

The porosity of the membrane has a large influence on the flow rate of the liquid and sensitivity of the detection test. The larger the pore size of the membrane, the faster the flow rate for a given liquid. As the flow rate increases, the interaction time available between the target molecule in the sample and the receptor immobilized on the reaction membrane decreases, thus decreasing detection test sensitivity. Additionally, larger pore sizes provide less surface area for immobilizing the receptor molecule, which is another parameter attributable to decreased detection test sensitivity. For the detection device of the present invention, the porosity of the membrane is preferably in the range of about 0.1 to about 12 microns, and more preferably about 0.45 to 3 microns. The porosity of the membrane preferably varies from about 0.2 to about 12 microns.

The wicking power of the membrane may also affect detection test sensitivity and depends on the thickness and nature of the membrane material. Wicking power can be measured as the migration of a standard solution through a certain distance per unit time. Often times, selecting a membrane having a relatively low wicking power can increase detection test sensitivity. In addition to porosity, the diameter and thickness of the reaction membrane may affect detection test sensitivity and therefore, must be considered. The depth or thickness of the membrane is selected so that an adequate amount of vaccinia viral lysate can be immobilized to capture the anti-vaccinia virus antibody. However, the thickness should not be so great as to cause undue delay of the passage of the fluid sample through the membrane.

The thickness of the reaction membrane, which is the distance between the upper and lower surfaces of the reaction membrane will range from about 0.05 mm to about to 3.0 mm, and more commonly from about 0.1 to about 1.0 mm. It has been found that when the thickness of the reaction membrane is greater than about 0.1 mm, and preferably in the range of about 0.2 mm to about 1.0 mm, higher sensitivity can be achieved. Additionally, a thicker reaction membrane may allow more vaccinia viral lysate to be available for binding to anti-vaccinia virus antibody, thereby providing a further increase in detection test sensitivity. It is believed that prior art devices which have relatively thin reaction membranes, such as nitrocellulose membranes less than 0.1 mm thick which are not paper-backed, tend to allow the sample to flow sideways across the reaction membrane rather than downwards through the middle of the reaction membrane.

However, as a presently preferred embodiment, the reaction membrane comprises paper-backed nitrocellulose, or other types of nitrocellulose membranes with similar characteristics. The preferred embodiment of the present invention comprises a nitrocellulose membrane backed with porous paper similar to filter paper. A representative example is commercially available under the trade name BAC-T-KOTE by Schleicher and Schuell. This preferred membrane is substantially more durable than nitrocellulose alone and can be employed without any other support component. Also, it provides enhanced sensitivity to the reaction. Also, polyester supported nitrocellulose may be used such as supplied under the name NITROPLUS by Micron Separation, Inc.

The term “reaction zone” is intended to include the porous material to which the vaccinia viral lysate and other molecules employed in the detection test are bound as well as additional porous supporting material, if any, that forms the lower surface of the reaction zone. For example, a preferred reaction zone comprises a sheet of nitrocellulose backed with a porous paper. Commercially available porous polyester supported nitrocellulose can also be used. A representative example of paper-backed nitrocellulose is commercially available from EY Laboratories Inc. (San Mateo, Calif.; Cat. Nos. PBNC15-1, PBNC15-10, PBNC15M-1, and PBNC15M-10). This preferred material is substantially more durable than nitrocellulose alone and can be employed without any other support component. This allows for easier handling and device assembly. Additionally, it has been found that detection devices employing paper-backed nitrocellulose for the reaction zone have enhanced sensitivity in most instances.

3.2 Immobilization of the Capture Reagent

In a typical system, the vaccinia viral lysate is immobilized on the porous membrane of the reaction zone which will specifically bind to any anti-vaccinia virus antibody present in the fluid sample being screened. The vaccinia viral lysate can be immobilized directly or indirectly onto such materials, such as nitrocellulose, by either absorption, adsorption, or covalent bonding. When a test sample suspected of containing anti-vaccinia antibody is applied to the reaction zone containing the immobilized vaccinia viral lysate, it becomes non-diffusively bound to the reaction zone. Thus, by appropriate application of a fluid sample suspected of containing anti-vaccinia antibody, a high concentration of the target analyte can be obtained in a well defined region within the center of the reaction zone. In appropriate cases, the vaccinia viral lysate may be coated on the upper surface of the reaction zone or entrapped within the matrix of the porous material of the reaction zone. Therefore, as used herein, the term “immobilized” is intended to embrace any means for fixing the capture reagent to the porous material.

A first step of the present method is to immobilize the capture reagent within a finite zone of the reaction zone. Immobilization can be accomplished by methods such as adsorption, absorption, evaporative deposition from a volatile solvent solution, covalent bonding between the capture reagent and the reaction membrane, or immunological immobilization. Covalent bonding may, for example, involve bonding the capture reagent to the reaction zone through a coupling agent, such as a cyanogen halide, e.g. cyanogen bromide or by the use of gluteraldehyde, as described by Grubb, et al. in U.S. Pat. No. 4,186,146. Immunlogical immobilization to the reaction membrane may be by absorption, or by covalent linkage, directly, or through a linker of sorts well-known to those skilled in the art. Suitable methods of carrying out these procedures are given, for example, by Iman and Hornby in Biochemical Journal (Volume 129; Page 255; Campbell, Hornby, and Morris in Biochem. Biophys. Acta (1975), Volume 384; Page 307; and Mattisson and Nilsson in F.E.B.S. letters, (1977) Volume 104, Page 78. See also, for example, U.S. Pat. Nos. 4,376,110 and 4,452,901. In addition, chemically pretreated materials suitable for coupling antibodies can be purchased commercially.

Immunological immobilization is preferred for the practice of the present invention wherein the porous reaction membrane is impregnated with a vaccinia viral lysate by way of absorption using a dispenser/printer technique (BioDot, California, USA). The technique essentially involves applying the lysate to the membrane by spraying it directly onto a porous reaction membrane. The above technique is most readily achieved using a commercial printing device termed a BIOJET QUANTI 3000, and provides a stream of the capture reagent under a variety of conditions, and at varying stream widths. Using this technique, it is possible to rapidly deposit a series of lines, or other discrete patterns on the reaction membrane. Preferably, the vaccinia viral lysate is deposited in a discrete test zone having an area substantially smaller than that of the entire surface area of the porous material. In addition, it is preferred that the pattern is in the form of a single discreet line to enhance the visibility of the result.

3.3 Control Zone

In addition to the capture reagent, a defined area of the exposed reaction zone may also contain a control molecule. In this regard, color development at the test site may be compared with the color of one or more standards controls to determine whether the reagents are stable and the test is performing properly. In general, when testing for the presence of anti-vaccinia antibody, the detection device will have a built-in control of an antibody directed to human immunoglobulin G (IgG), IgM, IgE, or IgA. Thus when a fluid test sample (e.g. plasma or serum) is added to the diagnostic device, immunoglobulin will bind to the control region regardless of whether or not analyte happens to be present in the sample. For example, a suitable control may be established by using Protein A which is disclosed in U.S. Pat. No. 5,541,059 (Chu). Other suitable controls are well known in the art.

3.4 Blocking the Reaction Zone

As noted above, the vaccinia viral lysate, and the optional use of controls, are typically applied only to defined regions of the exposed surface of the reaction zone. The vaccinia viral lysate will be applied to a defined region within the center of the reaction zone such that the perimeter of the exposed surface of the reaction zone will not have any lysate bound thereto. In this regard, the remainder of the porous material or membrane from which the zone is made can be treated with a blocking composition that prevents the target substance and other components of the sample from non-specifically binding to the reaction zone. The use of a good quality paper-backed nitrocellulose may make a blocking step unnecessary in some assays. However, if a blocking step is needed, a common blocking solutions comprising bovine serum albumin (BSA) or other proteins which do not interfere with or cross-react with reagent materials of the detection test can be used. BSA is usually used in amounts from about 1 to 10%.

The blocking treatment typically occurs after the detection device has already been assembled and the vaccinia viral lysate is immobilized to the reaction zone. A sufficient amount of blocking composition which will cover the exposed surface of the reaction zone is applied. After the blocking composition has dried, the detection device is ready for use.

3.5 Absorbent Zone

The sensitivity of reaction-membrane type immunoassays (i.e. the ability to detect very low levels of target substance) can be increased if the sample is concentrated through the reaction zone. With some devices, concentration of the sample through a reaction zone is achieved by having an absorbent material, or pad, beneath the reaction zone that draws the sample, which is added to the surface of the reaction zone, through to the absorbent material below. The absorbent zone can be generated from any material capable of wicking fluid by way of capillary action, such as cotton or paper. Membrane-based lmmunoassays that utilize various absorbent materials to concentrate sample are exemplified In U.S. Pat. Nos. 5,185,127, 5,006,464, 4,818,677, 4,632,901, and 3,888,629.

An absorbent material is situated underneath the lower surface of the reaction zone so as to be In direct fluid communication with the reaction zone. Thus, the upper surface of the absorbent material is immediately adjacent to the lower surface of the reaction zone. Fluid communication contact involving direct physical contact of the absorbent material with the reaction zone may include the separation of a portion of the absorbent material from the reaction zone by an intervening spacer layer, thereby essentially defining a test zone. Although not critical to the performance of the apparatus, the spacer layer also serves to hold the porous membrane of the reaction zone and permit detection test reagents to flow uniformly from the upper surface down to the lower surface of the detection test apparatus. The spacer layer may be made of any rigid or semi-rigid porous material that does not bind or interact with detection test reagents used in conjunction with the invention. In embodiments of the invention where ease of manufacture and reduced costs are desired, the upper surface of the absorbent material is typically immediately adjacent the lower surface of the reaction zone.

The selection of material for the absorbent zone is not critical and a variety of fibrous filter materials can be used, including one or more layers of the same or different materials, providing that the material selected is compatible with the analyte and the detection test reagents. Any conventionally employed absorbent material that is capable of drawing or wicking fluid through a porous membrane, such as for example, by capillary action, can be used in the present invention. The absorbent material should be capable of absorbing a volume of fluid sample that is equivalent or greater than the total volume capacity of the material itself. Useful known materials include cellulose acetate fibers, polyester, polyolefin or other such materials. The absorbent material provides a means to collect the sample by providing uniform “suction” to deliver the sample from the well, through the reaction zone, and down into the absorbent material. Thus, the absorbent body also acts as a reservoir to hold the sample, and various reagents that are used when the detection test is performed. Accordingly, when used In tests where relatively large volumes of fluid are used, the absorbent material should have high absorbent capacity so as to prevent or minimize the possibility of back-flow of sample and reagents from the absorbent body back into the reaction membrane.

As with the reaction zone material, the wicking power of the absorbent material can be an important parameter. Wicking time is defined in terms of the time required for water to travel a defined distance through the absorbent paper and Is related to the basis weight, thickness, and composition of the paper. Wicking power can vary greatly from one material to the next. Thus, the properties of the analytical device and flow rate of sample and reagents can be modified by varying the absorbent material used.

3.6 Blood Separation Zone

To facilitate the detection of anti-vaccinia virus antibody in a whole blood sample, an alternate embodiment of the present Invention provides a test unit capable of receiving and separating the fluid portion of a whole blood sample from the red blood cells (RBC) featuring a blood separation zone contiguous with the reaction zone. The blood separation zone functions to selectively retain cellular components (i.e. red blood cells) contained within the whole blood sample and deliver the remaining components of the RBC-free fluid portion of the blood sample, including any anti-vaccinia virus antibody, to the reaction zone for eventual analysis. This particular feature is useful in preventing any interference during visualization of a color reaction for the detection of anti-vaccinia virus antibody and avoids the necessity of obtaining a preliminary extraction of serum or plasma In settings where proper equipment to perform such a procedure is unavailable.

Various methods for the separation of blood cells from the fluid portion of blood are described using separation coatings, erythrocyte aggregating and agglutinating agents, materials having asymmetric pore sizes, polymer-containing matrixes, and multilayer systems, to name a few, e.g. U.S. Pat. No. 3,768,978 to Grubb et al., U.S. Pat. No. 3,902,964 to Greenspan, U.S. Pat. No. 4,477,575 to Vogel et al., U.S. Pat. No. 4,594,372 to Zuk, U.S. Pat. No. 4,753,776 to Hillman et al., U.S. Pat. No. 4,816,224 to Vogel et al., U.S. Pat. No. 4,933,092 to Aunet et al., U.S. Pat. No. 5,055,195 to Trasch et al., U.S. Pat. No. 5,064,541 to Jeng et al., U.S. Pat. No. 5,076,925 to Roesink et al., U.S. Pat. No. 5,118,428 to Sand et al., U.S. Pat. No. 5,118,472 to Tanaka et al., U.S. Pat. No. 5,130,258 to Makino et al., U.S. Pat. No. 5,135,719 to Hillman et al., U.S. Pat. No. 5,209,904 to Forney et al., U.S. Pat. No. 5,212,060 to Maddox et al., U.S. Pat. No. 5,240,862 to Koenhen et al., U.S. Pat. No. 5,262,067 to Wilk et al., U.S. Pat. No. 5,306,623 to Kiser et al., U.S. Pat. No. 5,364,533 to Ogura et al., and U.S. Pat. No. 5,397,479 to Kass et al..

In a preferred embodiment, the blood separation zone is an elongate or rectangular strip of porous material having Intrinsic physical properties which enable it to preferentially and sufficiently entrap or retain the red blood cells In the sample within the blood separation zone. A first end of the blood separation zone located a short lateral distance from the reaction zone, defines a region for receiving the whole blood sample during the initial stage of the detection test. A second end of the blood separation zone overlaps slightly with the reaction zone, so that it is in direct fluid communication with the reaction zone, thereby promoting the movement of the RBC-free fluid portion of the blood sample from the first end of the blood separation zone to the reaction zone for eventual analysis. The blood separation zone and the reaction zone must contact one another In order to ensure optimal transfer of the sample from one zone to the other.

A variety of materials can be used-for the blood separation zone such as glass fiber, glass fiber/cellulose mixtures, cellulose, or other proprietary materials, including synthetic materials, e.g., nylon. Preferably, a permeable glass fiber matrix is employed as the blood separation material to facilitate the separation of red blood cells from whole blood. A variety of grades of different thicknesses and absorbencies of glass fiber materials are commercially available to facilitate blood separation and include, for example, GF-24, GF-25, and #33, available from Schleicher & Schuell (Keene, N.H., USA); G143, G144, and G167, available from Ahistrom (Mount Holly Springs, Pa., USA); GFQA30VA, GF/P 30, GF/DE 30, GF/SE 30, GF/CM30VA, GF/CM 30, F 075-14, F487-09, GF DVA, GFVA 20, and GD-2, available from Whatman (Fairfield, N.J., USA).

Useful glass fiber/cellulose mixture materials include F255-07 90 glass/10 cellulose, F255-09 70 glass/30 cellulose, F255-11 50 glass/50 cellulose, and F255-12 50 glass/50 cellulose, available from Whatman.

Useful cellulose materials Include 598, available from Schleicher & Schuell. Miscellaneous or other materials falling outside the above categories can also be used, including HemaSep V and Leukosorb; which article of manufacture according to the subject invention available from Pall BioSupport (Port Washington, N.Y., USA).

One useful nylon material Is Nylon 6.6 Transfer Membrane, which is commercially available under the tradename Biodyne B (Pall Specialty Materials, Port Washington, N.Y.). In addition, the material known as “PlasmaSep”, available from Whatman, can be used.

Although the shape and dimensions of the blood separation zone are not critical, preferably It has a narrow rectangular form and dimensions suitable for allowing efficient removal of a substantial amount of red blood cells from the whole blood sample during migration of the fluid portion of the sample from the first end to the second end of the zone. Thus, in effect, while a narrow rectangular shape is preferred to channel fluid portion of the blood sample to the reaction zone, the dimensions may vary depending on the Intrinsic properties (e.g. absorbency, migration rate, etc.) of the material selected for the blood separation zone. In a preferred embodiment, the blood separation zone is made using the glass fiber material F487-09, available from Whatman, having dimensions between about 4 and 7 mm in width, between about 10 and 15 mm in length, and between about 0.2 mm and 1.0 mm in thickness. More preferably, the blood separation material is about 7 mm in width by about 10 mm in length and about 0.5 mm in thickness. These dimensions are optimized to be capable of receiving and separating the total volume of a whole blood sample, e.g. two drops of blood.

The blood separation material preferably has a rigid or semi-rigid carrier or backing affixed to its lower surface to provide support and reduce seepage of the RBC-free fluid portion of the whole blood sample while it migrates towards the reaction zone. Suitable materials for use as a carrier or backing include, for example, hydrophobic materials such as polycarbonate, polyethylene, Mylar, polypropylene, vinyl, cellophane and polystyrene, etc. as well as water-proofed or water-resistant cardboard or similar materials The carrier or backing may be affixed either directly or Indirectly to the blood separation material by means of an adhesive. Suitable adhesives are well-known in the art. The carrier may be of any shape and of almost any size which may conveniently be handled. However, the carrier is preferably similar in shape and size to the blood separation material. In a preferred embodiment, the carrier is formed as an elongate or rectangular strip having a length and width similar to or the same as the blood separation material.

4.0 Post-Filter Unit

As discussed above, the diagnostic device of the present invention comprises, as a second member, a post-filter unit comprising a label zone permeated with a dried indicator reagent.

The selection of the material for the label zone is not critical and can be any suitably absorbent, porous or capillary possessing material through which the push buffer and resolubilized indicator reagent may be transported by wicking action. The criteria of selection is that the material allow for the resolubilization and mixing of the dried indicator reagent upon addition of the push buffer, as well as initiate the transfer of the buffer and freshly dissolved indicator reagent to the reaction zone of the test unit.

Natural, synthetic, or naturally occurring materials that are synthetically modified, can be used as a filter medium including, but not limited to: cellulose materials such as paper, cellulose, and cellulose derivatives such as cellulose acetate and nitrocellulose, fiberglass, cloth, films of polyvinyl chloride, and the like. A preferred filter medium is nitrocellulose, however, the material should be chosen for its ability to premix the push buffer with the resolubilized indicator reagent. Moreover, the fluid flow through the filter medium should be laminar as opposed to turbulent flow characteristics which allows the initial mixing of the buffer with the indicator reagent. If nitrocellulose is used as the filter medium, then a preferred material is glass fiber filter paper because it enables the mixing and transfer of resolubilized indicator reagent to the reaction membrane.

4.1 Indicator Reagent The use of indicator reagents to detect the presence of a target analyte in a test sample is well known in the art. Depending on the type of diagnostic assay employed, the label employed in the indicator reagent is conjugated to a specific binding reagent that will directly, or indirectly, bind to anti-vaccinia antibody. Formation of an indicator reagent between a specific binding reagent and a label may be any of the conventional types including metal complex labels, radioactive labels, enzyme labels, fluorescent labels, radioactive labels, chemiluminesct labels, and the like.

An important consideration in the design of the rapid vaccinia antibody detection device is that the label chosen in the generation of the indicator reagent should give rise to a readily detectable signal, e.g. a strongly-coloured area easily detectable by the eye. Thus, an important preferred embodiment of the invention is the use of “direct labels”, attached to one of the specific binding members. Direct labels are well known in the art and highly advantageous for their use in rapid detection systems. Examples of direct labels include, but are not limited to metal sols, non-metal sols, dye sols, latex particles, carbon sol, and liposome contained colored bodies. Some of their advantages are that they can be used to produce a visually detectable signal without the need to add further reagents, are readily visible to the naked eye without the aid of instrumentation, and can be readily used in a diagnostic device since they are stable when stored in the dry state. With respect to the latter, their stability and immediate release on contact with a buffer reagent can be accomplished by the use of soluble glazes.

Non-metal sols, such as those of selenium, tellurium and sulfur may be produced according to the methods described in U.S. Pat. No. 4,954,452 (Yost, et al). Dye sol particles may be produced as described by Gribnau et al., in U.S. Pat. No. 4,373,932 and May et al., WO 88/08534, dyed latex as described by May, supra, Snyder, EP-A 0 280 559 and 0 281 327, and dyes encapsulated in liposomes by Campbell et al., U.S. Pat. No. 4,703,017. The use of polymerized dye materials in colloidal form for specific binding assays is also described by in U.S. Pat. No. 4,166,105 by Hirschreid which relates to labelled specific binding reagents reactive with specific antigens prepared by linking fluorescent dye molecules to analyte specific antibodies through polymers comprising reactive functional groups. Also of interest is U.S. Pat. No. 4,313,734 by Leuvering relating to metal sols, the disclosure of which is incorporated herein by reference; Leuvering, et al., “Sol Particle Immunoassay (SPIA)”, Abstract, Journal of Immunoassay, 1(1), pp. 77-91 (1980); Leuvering Dissertation (1984), Sol Particle Immunoassay (SPIA): The Use of Antibody Coated Particles as Labelled Antibodies In Various Types of Immunoassay; Uda et al., Anal. Biochem. 218 (1994), 259-264, DE-OS 41 32 133, page 3, lines 16-18, for applications as markers and Tang et al., Nature 356 (1992), 152-154; Eisenbraun et al., DNA and Cell Biology 12 (1993), 791-797. Furthermore it is also known that non-metallic colloidal particles such as carbon particles (van Amerongen, Anabiotic '92 (1993), 193-199) can also be used. Moeremans, et al., EPO Application No. 158,746 discloses the use of colloidal metal particles as labels in sandwich blot overlay assays. At present colloidal gold particles are used most frequently.

Among the direct labels, metallic sols are preferred, more preferably gold sol particles such as those described by Leuvering in U.S. Pat. No. 4,313,734. Leuvering discloses the use of metal sol particles as labels for in vitro determination of immunological components in an aqueous test medium. Specifically disclosed are immunoassay test kits for the detection of antigens or antibodies employing one or more labelled components obtained by coupling the component to particles of an aqueous sol dispersion of a metal, metal compound or polymer nuclei coated with a metal or metal compound having a particle size of at least 5 nm.

The metal sol particles to be used in accordance with the present invention may be prepared by methodology which is known. For instance, the preparation of gold sol particles is disclosed in an article by G. Frens, Nature, 241, 20-22 (1973). Additionally, the metal sol particles may be metal or metal compounds or polymer nuclei coated with metals or metal compounds, all as described in the Leuvering patent mentioned above. In this regard, the metal sol particles may be of platinum, gold, silver or copper or any number of metal compounds which exhibit characteristic colors.

4.2 Colloidal Gold Particles

Colloidal particles which are suitable as labels according to the invention include those which may be conjugated to specific binding reagents without interfering with the activity of such reagents or with other reagents or analytes.

Colloidal metal particles are particularly suitable as labels according to the present invention and include those particles which are comprised of metals or metal compounds selected from the group consisting of the metals platinum, gold, silver and copper and the metal compounds, silver iodide, silver bromide, copper hydroxide, iron oxide, iron hydroxide or hydrous oxide, aluminum hydroxide, or hydrous oxide, chromium hydroxide or hydrous hydroxide, lead sulfide, mercury sulphide, barium sulphate and titanium dioxide. Preferred colloidal metal particles include those made up of gold.

Colloidal gold particle markers are simple to use in comparison to other conventional markers. For example, they do not require instruments necessary for detection of other markers such as radioactive isotopes and unlike enzymes, they do not require the additional step of adding a substrate.

Colloidal gold particles may be produced according to methods generally known in the art. Of interest to the present invention are those references relating to the use of dispersions of colloidal particles in immunological assay procedures. Specifically, Frens, Nature, 241, 20-23 (1973) discloses methods for the production of gold sol particles of varying sizes through the reduction of gold chloride with aqueous sodium citrate. The colors of the visually detectable signal from the metal particle label is dependent upon the identity and particle size of the metal particle which may be controlled by varying the concentration of the reactants. For example, colloidal gold particles produce colors ranging from orange to red to violet depending upon the particle size of the sol.

The colloidal gold reagent is selected for its unusual properties: the ability to intensify color to the naked eye when concentrated on solid surfaces, the ability to minimally bind nonspecifically to solid surfaces, the ability to be prepared in relatively uniform particle sizes, and the ability to be easily lyophilized and resolubilized. Colloidal gold particles can be prepared in a number of ways through the reduction of tetrachloroauric acid which produces a variety of particle sizes ranging from 5 nm to 100 nm. The preferred particle sizes are from 15 to 20 nm. The colloidal gold particles can have an intermediary binder absorbed to its surface prior to the addition of the binding substance, but direct attachment is satisfactory. Absorbing the selected binding substance is achieved by carefully controlling concentrations, ionic strength and pH of the reaction mixture. The choice of method of producing the colloidal gold raw material or the method of attaching the binding substance are well known to those skilled in the art. After the labeling with colloidal gold is complete, the reagent is differentially centrifuged or filtered to control particle size. Particle sizing by gel filtration methods are also well known. The colloidal gold labeled reagent can be used as a colloidal suspension or as a lyophilized reagent with or without the presence of the aforesaid solid phase particles as an indicator reagent.

The resulting coated and stabilized colloidal metal particles may then be conjugated with various proteins. Any protein which may be subjected to freeze-drying or other forms of drying such as by incubator, air-drying and spray drying may be applied in the present invention. Exemplary of protein for use in the present invention includes, but is not limited to, polyclonal or monoclonal antibodies, antigen, lectin, protein A, protein G, bacterial, and the like. A preferred methods of detecting the presence of anti-vaccinia antibody is the utilization of a labeled Protein A colloidal gold conjugate.

For details and engineering principles involved in the synthesis of colored particle conjugates see Horisberger, Evaluation of Colloidal Gold as a Cytochromic Marker for Transmission and Scanning Electron Microscopy, Biol. Cellulaire, 36, 253-258 (1979); Leuvering et al, Sol Particle Immunoassay, J. Immunoassay 1 (1), 77-91 (1980), and Frens, Controlled Nucleation for the Regulation of the Particle Size in Monodisperse Gold Suspensions, Nature, Physical Science, 241, pp. 20-22 (1973). Surek, et al., Biochem. and Biophys. Res. Comm., 121, 284-289 (1984) discloses the use of protein A labeled colloidal gold particles for the detection of specific antigens immobilized on nitrocellulose membranes.

4.3 Drying Process—Sugar/Glazing Treatment

According to one important aspect of the invention, the indicator reagent is impregnated and dried within the thickness of the porous material defining the label zone of the post-filter unit, which may then be resolubilized by addition of the push buffer. Thus, by incorporating one of the detection test reagents in the device of the present invention, makes possible the reduction in the number of steps required in the detection test protocol by eliminating the addition and/or prior mixing of an indicator reagent.

In order to assist the free mobility of the indicator reagent when the label zone of the post-filter unit is moistened with the push buffer, the post-filter unit is pre-treated with a glazing material In the region to which the indicator reagent is applied. Glazing can be achieved, for example, by depositing an aqueous sugar or cellulose solution, e.g. of sucrose or lactose, on the relevant region of the post-filter unit, while avoiding the remainder of the filter unit, and air drying. The indicator reagent can then be applied to the glazed portion.

The glazing process involving the use of one or more sugars (e.g. glucose, lactose, trehalose and sucrose) is highly advantageous when employing the dried indicator reagent of the present invention in that the sugar serves (1) as a protein stabilizer, (2) to improve the long term stability of the dried indicator reagent, and (3) acts as a rapid releasing agent. According to a preferred embodiment of the invention, sucrose was determined to be the best sugar compared to others in the performance of the detection test because of (1) its solubility, ( 2) short period of drying, (3) the overall sensitivity of the detection test result, (4) its use as a preservative, and (5) it is economical to use.

5.0 Buffer Reagent

Conventional detection assays usually necessitate the use of two or more fluid reagents in order to perform various steps of the assay protocol including, for example, resolubilizing a dried indicator reagent, diluting a biological test sample, blocking the membrane surface where the assay reaction takes place, facilitating transport of critical reagents and/or washing unbound reactants from the reaction zone. Since each of these steps involves the mixing or preparation of different reactants, different formulations of liquid reagents are likely required due to differing pH, Ionic strength, additives, type and strength of buffer, temperature, etc. For example, the resolubilization process usually requires the use of a physiological buffer such as buffered saline or double distilled water, the blocking process uses a liquid reagent formulated with any number of animal serum albumins, gelatin or non-fat milk, and the washing and/or diluting process involves the use of a phosphate buffered saline containing different amounts of surfactant or detergent at neutral pH to remove any non-specific binding reactants. Moreover, in order to ensure that the user performs each step of the assay correctly using the appropriate liquid reagent, the reagents themselves must be clearly labeled and readily distinguished from one another, so as to avoid any possible confusion and user error.

An important aspect of the present invention overcomes the various problems described above by providing a push buffer which is a multifunctional in that it serves as a single buffer reagent for utilization in the aforementioned 2-step detection test procedure. The push buffer is formulated to serve as a combination resolubilization reagent of the dried indicator reagent, transport facilitating reagent of resolubilized indicator reagent from the label zone of the post-filter unit to reaction zone of the test unit, and washing reagent to remove unbound reactants from the reaction zone. Moreover, since the reaction zone of the present invention is already pretreated with conventional blocking agents following immobilization of the vaccinia viral lysate, the buffer formulation eliminates the need to include a non-specific blocking agent. In order to simplify the number of reagents and steps required to perform the detection test, the push buffer has been specially formulated to be used in conjunction with the dried indicator reagent. It is therefore, particularly advantageous to utilize the push buffer and dried Indicator reagent as a combined system since they allow optimal sensitivity and higher specificity to be achieved during performance of the detection test. Additionally, aggregation and inactivation of the indicator reagent in solution is avoided without sacrificing either the sensitivity or specificity of the detection test. A method of using the push buffer as provided by the present invention involves dropwise addition of the buffer to the post-filter unit in a single application of the 2-step detection test to resolubilize the dried indicator reagent. A kit containing the push buffer as a component is also provided.

Accordingly, the present invention provides an improved buffer formulation for use with the rapid vaccinia antibody detection device, comprising a biological buffer (e.g. phosphate buffer) in a sufficient concentration to maintain the pH between 7.0 to 10.0; at least one surfactant (e.g. Triton®X-100); a high molecular weight polymer (e.g. polyvinylpyrrolidone); a pH stabilizer (e.g. trizma hydrochloride); an ionic salt (e.g. NaCl); and a calcium chelator (e.g. EDTA); all at effective concentrations. The buffer also can include sodium azide and thimerosal as preservatives at an effective concentration to reduce bacterial and microbial growth and thus, prolong the shelf life of the reagent.

The push buffer composition of the invention can include a conventional buffer such as a phosphate buffer, MES (morpholino-ethanesulfonic acid) buffers, BIS-TRIS buffers, citrate buffers, TRIS-HCl buffers and borate buffers, at an effective concentration which can range from about 10 to 100 mM, preferably in the range of from about 10 to 30 mM, and most preferably about 20 mM. The preferred buffer is a phosphate buffer, preferably comprising sodium phosphate, monobasic and sodium phosphate, dibasic, at concentrations such that the effective concentration of buffer is achieved. The pH of the buffer of the present invention can range from a pH of about 7.0 to a pH of about 10.0.

The biological detergents (surfactants) used in the present Invention can include non-ionic surfactants, anionic surfactants, zwitterionic surfactants and cationic surfactants. The Non-ionic detergents useful in the invention include polyoxyethylene sorbitan monolaurate (Tween®20), polyoxyethylene sorbitan monooleate (Tween®80), polyoxyethylene ethers(Triton®, Brij®) and octylphenel ethylene oxide (Nonidet®). Preferably, non-ionic detergents are used. The most preferred non-ionic detergent is Triton®X-100. Non-ionic detergent acts as a dispersing agent to reduce the non-specific binding of antibodies/antigens to the reaction membrane which may occur as a result of target analyte adhering to the solid phase due to a non-specific reaction, thereby increasing the background of the assay. Although biological detergents reduce the event of such binding caused by nonpolar or hydrophobic interactions, non-ionic detergents are preferred for their ability to reduce non-specific binding while avoiding the inhibition of specific binding. Effective concentrations of the biological detergent range from about 0.005 to about 0.06%, (w/v) preferably range from about 0.01 to about 0.025%, and most preferably the concentration is about 0.017%.

Effective concentrations of sodium chloride range from about 0 to about 300 mM, preferably range from about 100 to about 200 mM, and most preferably the concentration of sodium chloride is about 150 mM.

Polyvinylpyrrolidone functions as a dispersing and suspending agent while additionally preserving the binding capacity of antibodies by blocking non-specific sites on the reaction membrane. Effective concentrations of polyvinylpyrrolidone (PVP) in the buffer of the invention range from about 0.1 to about 3.0%, (w/v) preferably range from about 0.5 to about 2.5%, and most preferably the concentration is about 1.4%. The high molecular weight polymer selected for use in the invention can include PVP having molecular weights of from about 10 kD to about 1500 kD, dextrans with molecular weights ranging from about 10 kD to about 2000 kD, polyethylene glycols (PEG) having molecular weights in the range of from about 200 D to about 10,000 D, polyvinyl alcohol having a molecular weight of about 10,000 D to about 100,000 D, polybrene (hexadimethrine bromide), methylcellulose, gum acacia, protamine sulfate, merquat, celquat and magnafloc, provided at an effective concentration. The preferred high molecular weight polymer is PVP; most preferably, PVP-40 is provided at an effective concentration.

The addition of a calcium chelator, preferably, preferably EDTA, to the push buffer composition is essential to prevent the possible clotting of a test sample through calcium-depleting action. The effective concentration of EDTA is from about 10 to 100 mM.

The pH stabilizer functions to maintain the pH of the buffer within a range of about pH 7.0 to 10.0. An exemplary pH stabilizer includes trizma hydrochloride, although other known stabilizers may also be useful in this composition. The effective concentration of trizma hydrochloride is from about 0.02 to 0.05 mM.

6.0 Housing

In general, the detection test composite comprising the test unit and the post-filter unit can be housed in a suitable container to form a detection apparatus. Preferably, the container should safeguard the solid phase materials and dried indicator reagent from contamination and to provide ease and convenience in handling of the detection test device. Moreover, the container should be leak-proof thereby ensuring containment of fluids and their safe disposal after use.

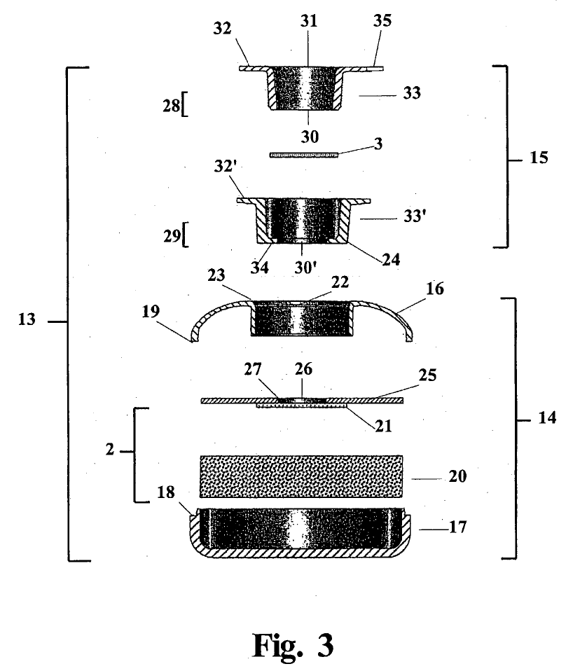

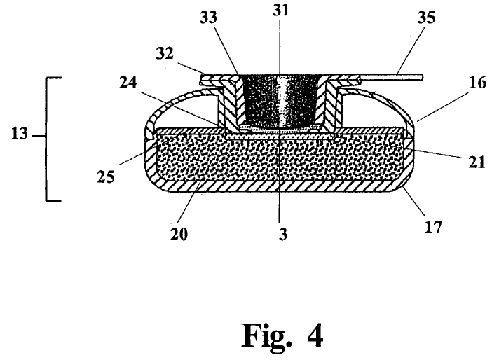

The apparatus 13 illustrated in FIGS. 3 and 4 provides a representative example of the type of container that can be included in a test kit which incorporates the flow-through detection test of the present invention. The apparatus 13 comprises two detachable components, namely the test cartridge 14 and the post-filter cap 5, which are vertically and spacially distinct to one another when placed in transient communication during the detection test protocol. The housing is capable of maintaining the layers of the test unit under compression so as to provide continuous and uniform contact therebetween and so that liquid will flow uniformly through the apparatus 13. The housing will be made of an inert material conveniently being any of a variety of disposable commercial plastics which may be molded, for example, polyethylene, polypropylene, styrene, ABS, polyacrylate, polystyrene, or the like.

Although the two components of the apparatus 13 have the particular configuration and dimensions depicted therein, any other appropriate design or modifications may be employed so long as the components are still capable of being transiently connected to one another in a single movement during the detection test protocol. The means of connecting the two components is not critical so long as so that they are properly aligned to effect optimal fluid communication with one another upon interconnection of the post-filter cap 15 with the test cartridge 14. For example, according to the design shown in FIGS. 3 and 4, the post-filter cap 15 may be frictionally fitted to the reservoir 22 of the test cartridge 14. Although not illustrated therein, the post-filter cap 15 may be optionally hinged to the test cartridge 14 to avoid possible lost or misplacement of the two components. On the other hand, the two components could be slidably and reversibly disposed to one another in a single horizontal movement providing the post-filter unit is engaged in proper alignment above the test unit. In this particular Instance, proper alignment of the two components may be achieved through the use of guide rails, or projections designed to align with recesses formed in the device or housing, additionally acting as an interconnection means for the two components.

The precise dimensions of the housing are not essential to the function of the detection test apparatus, but in general, the apparatus will be of a size convenient for transport, manipulation, and assembly. The housing will generally have a length in the range of about 2 to 5 cm; preferably 3.5 cm. The width will be in the range of about 1 to 3 cm, preferably 2.5 cm. The height of the housing will be in the range of about 0.5 to 5 cm, preferably 1.3 cm.

FIG. 3 provides an exploded view of the apparatus 13 comprising the test unit 2 and the post-filter unit 3, while FIG. 4 provides an enlarged vertical cross-sectional view of the fully assembled apparatus 13. The apparatus 13 of the present invention comprises two separate components in its fully assembled form, namely the test cartridge 14, which contains the test unit 2, and the post-filter cap 15, which contains the post-filter unit 3. The test cartridge 14 and the post-filter cap 15 are designed to be connected to one another briefly during the detection test protocol. The apparatus 13 is intended to be simple in design and construction, and can be manufactured using readily available materials.

As shown in FIG. 3, the test cartridge 14 of the apparatus 13 houses the test unit 2 which comprises both a top member 16 and a bottom member 17. The outer perimeter of the bottom member 17 has a slightly indented ridge 18 which allows it to be fitted and interconnected with the rim 19 bordering the top member 16 to form the assembled test cartridge 14. It will be appreciated by those skilled in the art that while the test cartridge 14 shown in FIGS. 3 and 4 has a rectangular shape, it is not limited to this particular configuration so long as it can be adapted to hold the absorbent material, or pad 20, in direct contact with the porous reaction membrane 21.

Contained within the top member 16 of the test cartridge 14 is a reservoir 22 which is in direct alignment with the exposed reaction membrane 21 of the test unit 2. The reservoir 22 (a) provides access to the reaction zone 5 for introducing the fluid sample, (b) provides operable attachment of the post-filter cap 15 for introduction of the push buffer, and (c) permits viewing of the “indicator” zone(s) on the reaction membrane 21 following removal of the post-filter cap 15, i.e. detect the color, or fluorescence, or other signal, in the indicator zone(s). As depicted In the drawing, the upper surface surrounding the reservoir 22 is slightly curved and extended downwards so as to form a cup-like receptacle which terminates at, and firmly engages a portion of the reaction membrane 21. In this way, the amount of test sample introduced into the reservoir 22 cannot bypass any components of the apparatus 13. The configuration of the inner wall 23 and the dimensions of the reservoir 22 are selected so that the reservoir 22 can connect to and be in operable association with the post-filter cap 15 during the detection test protocol. Preferably, both the reservoir 22 and the post-filter cap 15 have a funnel shape configuration. Thus, when the reservoir 22 and the post-filter cap 15 are in the operating position and the push buffer is applied to the filter cap 15, this configuration will permit a large amount of the buffer to contact and pass through a small amount of surface area of the reaction membrane 21. Thus, by selectively matching the size of reservoir 22 with the post-filter cap 15, the operation of the apparatus 13 can be simplified so that, for example, the push buffer 12 can be delivered to the reservoir 22 in a single step of the detection test procedure.

According to the embodiment shown In FIGS. 3 and 4, the post-filter cap 15 is detachable affixed to the reservoir 22 of the test cartridge 14 by means of a friction fit between the inner wall 23 of the reservoir 22 and the external wall 33′ of the filter cap 15. Such other means for detachable affixing the post-filter cap 15 to the test cartridge 14 can be used. In addition, the height of the external wall 33′ of the post-filter cap 15 is slightly less than the height of the inner wall 23 of the reservoir 22 so that when the filter cap 15 is affixed to the reservoir 22, the base 24 of the filter cap 15 terminates immediately above, but not touching, the reaction membrane 21. The dimensions of both the reservoir 22 and the post-filter cap 15 can be varied considerably without affecting the performance of the apparatus 1.3, although the following approximate dimensions have been determined as satisfactory: reservoir 22—1.5 cm top and bottom diameters and 0.6 cm deep; post-filter cap 15—0.9 cm bottom diameter, 1.1 cm top diameter, and 0.5 cm deep.

As described above, the test unit 2 of the present invention comprises a porous reaction membrane 21 and an absorbent pad 20, whereby the lower surface of the reaction membrane 21 is supported by the upper surface of the absorbent pad 20. The reaction membrane 21, which contains vaccinia viral lysate as capture reagent, essentially defines the reaction zone in which various specific binding reactions take place during the detection test. As previously described, the reaction membrane 21 can be fabricated from a number of biologically inert, porous materials.

Positioned directly underneath the lower surface of the reaction membrane 21, and in fluid communication therewith, is an absorbent pad 20 defining the absorbent zone. In embodiments of the invention where ease of manufacture and reduced costs are desired, the entire upper surface of the absorbent pad 20 is typically immediately adjacent the lower surface of the reaction membrane 21. The test unit 2 may optionally include a separating means between the reaction membrane 21 and the absorbent pad 20 which will generally be incapable of binding the anti-vaccinia virus antibodies. According to the embodiment shown in FIG. 3, the separating means in the form of a spacer layer 25 substantially isolates the reaction membrane 21 from the absorbent pad 20. Although not critical to the performance of the apparatus 13, the spacer layer 25 serves to hold the porous reaction membrane 21 and permit test reagents to flow uniformly from the upper surface down to the lower surface of the detection test apparatus 13. The spacer layer 25 may be made of any rigid or semi-rigid porous material that does not bind or interact with test reagents used in conjunction with the invention. Exemplary of materials for the spacer layer 25 are fiberglass, paper, hydrophilic polypropylene, and cellulose; preferably the spacer layer 25 is made of H-HDC (Pall). The thickness of the spacer layer 26 will generally be in the range of about 0.1 mm to 1 mm.

The spacer layer 25 has an aperture 26 defined by a rim 27 which has similar circumferential dimensions and/or shape as the porous reaction membrane 21 thereby enabling the upper and lower surfaces of the reaction membrane 21 to be accessible when the membrane 21 and the spacer layer 25 are sealed together to form a press-fit piece. Referring to FIG. 3, which depicts one embodiment of the apparatus 13, a portion of the reaction membrane's 21 upper surface is fully exposed so that when the detection test is performed, the fluid test sample and the test reagents can be added directly to the reaction membrane 21. The reaction membrane 21 is sized to completely cover the aperture 26. Preferably the reaction membrane 21 will be the same shape as the aperture 26 and but sized slightly larger than the aperture 26 so that it can be sealed to the lower surface of the spacer layer 25 at the periphery of the aperture 26. However, the shape of the reaction membrane 21 and the shape of the aperture 26 can differ and are not limited to the configuration shown in FIG. 3. Thus, in combination, the rim 27 surrounding the aperture 26 and the exposed upper surface of the reaction membrane 21 define a test zone. Moreover, after the test cartridge 14 of the apparatus 13 is assembled, the absorbent pad 20 is still capable of contacting the lower surface of reaction membrane 21 located directly beneath the reaction membrane 21. The dimensions of the spacer layer 25 and the absorbent pad 20 are chosen to fit cooperatively within the base of the test cartridge 14, thereby ensuring that the absorbent pad 20 is in proper alignment and fluid communication with the lower surface of reaction membrane 21. Thus, the surface area of the upper surface of the absorbent pad 20 will usually be greater than that of the reaction membrane 21, but similar to that of the spacer layer 25.

The absorbent pad 20 is selected to have a capillary pore size so as to induce flow of the fluid test sample through the reaction membrane 21 without the use of external means. Thus, conveniently, the absorbent pad 20 serves to both promote and direct the flow of reagents through the porous reaction membrane 21. The absorbent pad 20 is of sufficient size and composition so that it is capable of absorbing excess sample, indicator reagent and buffer. The material from which the absorbent pad 20 is fabricated may be any non-porous wettable material that is substantially inert to the test reagents employed in the performance of an detection test. The absorbent pad 20 will be of essentially the same dimensions and shape as the spacer layer 25 which holds the porous reaction membrane 21. The precise thickness of the absorbent pad 20 is not essential to the function of the present invention, generally ranging from about 2 to 10 mm.

The second component of the apparatus is the funnel-shaped post-filter cap 15 which readily accommodates a sufficient amount of the push buffer needed to perform the detection test in a single application. The post-filter cap 15 comprises the post-filter unit 3 and inner 28 and outer 29 sleeves being open-ended at both the top and bottom. The bottom opening 30,30′ of sleeves 28 and 29 is sized to achieve the flow rate desired for the detection test in question. The opening of the sleeves can conveniently have a diameter in the range of 12.6 to 15.2 mm. Preferably the opening 30,30′ diameter is 9.5 mm.

In the assembled form, the post-filter cap 15 comprises a funnel 31 having at its top outwardly extending flanges 32, 32′ and depending sidewalls 33,33′. The depending sidewalls 33 of the outer sleeve 29 end at a base 24. The opening 30 in the base 24 allows a stream of fluid traveling through the funnel 31 to flow into the test cartridge 14. The post-filter unit 3 of the present invention is securely held in the base 24 of post-filter cap 15 by the inner 28 and outer sleeves 29 of the post-filter cap 15. An inner collar 34, integrally formed at the base 24 of the outer sleeve 29, is capable of supporting the post-filter unit 3 so that when the inner sleeve 28 is frictionally fitted inside the outer sleeve 29, the post-filter unit 3 will be held permanently in place.

The post-filter unit 3 comprises a filter medium impregnated with dried indicator reagent which defines the label zone. The dried indicator reagent is resolubilized and transported by the push buffer to the reaction membrane 21 following addition of the buffer to the post-filter cap 15. The selection of the filter medium for the post-filter unit 3 is not critical to the invention and can be any suitably absorbent, porous or capillary possessing material through which the push buffer and resolubilized indicator reagent may be transported by wicking action. The criteria of selection is that the material allow for the resolubilization and mixing of the dried indicator reagent upon addition of the push buffer, as well as initiate the transfer of the buffer and freshly dissolved indicator reagent to the reaction membrane 21 of the test unit 2.

For convenience of manipulation in using the apparatus 13, a handle 35 is secured to the extending flanges 32,32′ of post-filter cap 15 so that when the filter cap 15 is affixed to the reservoir 22, it extends slightly beyond the boundary of the reservoir 22 for ease of removal of the post-filter cap 15 from the test cartridge 14.

A representative example of a modified version of the test cartridge of the Invention incorporating a blood separation zone contiguous with the reaction zone for the detection of analyte in a whole blood sample is illustrated In FIGS. 6A and 6B.

As shown in FIG. 6A, the test cartridge Is provided with a top member 16 constructed and adapted to fit snugly with a bottom member 17. In this particular embodiment, the top member 16 of the test cartridge defines a first opening or internal recess therethrough in the form of a reservoir 22. The reservoir 22 serves to (a) provide operable attachment of the post-filter cap for introduction of the push buffer, and (b) permit viewing of the “indicator” zone(s) on the membrane following removal of the post-filter cap, i.e. detect the color or fluorescence, or other signal, in the indicator zone(s). Thus, the configuration and dimensions of the reservoir 22 are selected on the basis that it can be operably connected to the post-filter cap to enable transient fluid communication between the label zone of the post-filter unit and the reaction zone of the test unit.

Spaced a short lateral distance from the reservoir 22, the top member defines a second opening therethrough In the form of a reservoir 104 which may, as shown, have beveled sides, or may be in any shape or size or configuration of convenience which will sufficiently direct and provide access to the first end of the blood separation zone upon application of a whole blood sample. After introducing a whole blood sample to the reservoir 104 and allowing for a short incubation time to enable sufficient separation and migration of the RBC-free fluid along the blood separation zone to the reaction zone, the post-filter cap is operably attached to the reservoir 22 to enable completion of the 2-step detection test protocol so that a final determination for the presence of analyte can be made.

As shown in FIG. 6B, the bottom member 17 of the test cartridge provides a first base structure 105 having a plurality of supporting walls which serve as a solid enclosure for the absorbent pad and thus, is configured to receive and hold the absorbent pad securely in place. Additionally provided is a second base structure 106 having a plurality of protruding columns of the same height which serves as an elevated support for the blood separation zone. The position of the second base. structure 106 in relation to the first base structure 105, as well as its configuration, are such that when the blood separation zone is positioned within the bottom member 17, the blood separation zone is contiguous with and in direct planar horizontal alignment with the reaction zone. Although the base structure 106 depicted therein has a plurality of supporting columns and/or walls which serve to support the perimeter and centre of the blood separation zone, any number of configurations or strategies are possible as long as the blood separation zone is securely and correctly positioned In relation to the reaction zone when the test cartridge is fully assembled.

7.0 Test Kit

The rapid vaccinia antibody detection device of the present invention, which incorporates the test unit and post-filter unit, will typically be packaged in the form of a test kit. The kit will normally include the flow-through detection test device, preferably housed in a suitable container, the push buffer, a disposable plastic pipette and instructions for carrying out the detection test. Such instructions will generally describe the method for carrying out the detection test protocol including the relative amounts of test sample and push buffer to be added to the test unit and post-filter unit, respectively.

Preferably, the detection device is housed within a suitable container which comprises two detachable components, each component separately containing the test unit and the post-filter unit, which are arranged in a vertically and spacially distinct format. In particular, the design of the container should allow the test unit and post-filter unit to be operably connected to one another so that the reaction zone and the label zone can be placed in transient fluid communication with one another during the detection test protocol. The container housing the test unit should be capable of maintaining the layers of the test unit under compression so as to provide continuous and uniform contact therebetween so that liquid will flow uniformly through the apparatus. FIGS. 3, 4 and 6 provide a representative example of the type of container that can be included in a test kit which incorporates the flow-through format of the rapid vaccinia antibody detection device.

All publications and patent applications mentioned in this specification are indicative of the level of skill of those skilled in the art to which this invention pertains. All publications and. patent applications are herein incorporated by reference to the same extent as if each individual publication or patent application was specifically and individually indicated to be incorporated by reference.

The invention will be further understood from the following non-limiting examples. The following examples are provided to describe in detail some of the representative, presently preferred methods and materials of the invention. These examples are provided for purposes of illustration of the inventive concepts, and are not intended to limit the scope of the invention as defined by the appended claims.

### 8.0 EXAMPLES

The foregoing is a general description of the apparatus, method and reagents of the invention for the detection of anti-vaccinia virus antibody. Although dye sols, gold sols or coloured latex particles may be linked to Protein A to form the indicator reagent, the preferred visual label utilized in the example detection test will be colloidal gold particles. Using an apparatus comprising the test unit and post-filter unit, such as the one illustrated in FIGS. 3 and 4, and by performing the 2-step rapid detection test of the present invention, a determination of antibody against vaccinia virus in a whole blood or serum test sample can be made in less than three minutes.

8.1 Vaccination Complications

Certain groups of people are at high risk for complications if such a mass vaccination program were to be implemented. Immunodeficient individuals, such as HIV/AIDS patients, would be prone to Progressive Vaccinia (or vaccinia necrosum), a dermal complication resulting from exposure of immunocompromised individuals to the vaccinia virus (WHO, 2001). Specifically Progressive Vaccinia could prove to be deadly to an HIV-Infected Individual resulting from an accidental or inadvertent vaccination with the vaccinia virus or through direct contact with a person who had been recently vaccinated (Shanley, 2002). For example, vaccination of an HIV/AIDS patient with the faccinia virus could result in death within a few months following. Mortality rates associated with this condition were nearly 100% in the 1950's and early 60's but drastically decreased to 33% with the advent of vaccinia immune globulin (VIG) in 1969 (Kaufman, 2001). Such individuals will develop secondary lesions, which will progressively spread until death. As a result, knowing an inidividual's HIV status prior to small pox vaccination must be a mandantory requirement. The commercially available MedMira Reveal™HIV test will provide an efficient means to ensure that HIV testing can be provided immediately prior to vaccination whereas conventional laboratory testing for HIV can take days for results. Thus, it is recommended that the MedMira Reveal™Rapid HIV test should be used in conjunction with the smallpox vaccination program to eliminate the complications associated with vaccination.

The 114 million young Americans who have not been vaccinated against smallpox also fall into the high-risk group for HIV-infection (Henderson et al., 1999). The entire United States population of almost 300 million (2001) will require prevaccination screening for rapid diagnosis of HIV to aid in avoidance of the potential tragedy of “preventable death” due to the wrongful administration of the smallpox vaccine to immune-deficient individuals. In following the lead of the Americans, who are considered by many countries worldwide to maintain the highest of public health standards, other developed nations encompassing a population of approximately 1.2 billion (2001) may also implement this testing/vaccination algorithm.

8.2 Preparation of Vaccinia Virus Cell Lysate

The seed virus (V-CL) used in this particular preparation was obtained from Connaught Medical Research Laboratories In Toronto, Ontario. It is a standard strain of vaccinia virus produced through routine preparation of calf lymph.

Vaccinia virus preparation involves the infection of a monolayer of HeLacells with a 10⁻² dilution of frozen virus stock. The culture is then incubated for 2 to 3 days at 36° C. Once destruction of the HeLa cells is complete, the culture was subjected to a freeze/thaw cycle three times and then disrupted for 15 minutes in the cooled water bath of an ultrasonic vibrator. Cell debris is removed by low speed centrifugation (2,000 rpm for 20 minutes) and the resulting supernatant fluid is collected and stored at −20° C. in 1 mL volumes until used.

8.3 Rapid Flow-Through Membrane Application

The resulting supernatant containing the soluble viral lysates was diluted to 1:8 in carbonate buffer (pH 9.0) containing 0.5% sucrose and 5 mM NaCl. This preparation was then subjected to a 0.45 μm filtration process and the resulting clear fluid was used as the final cocktail for immunobinding application on a paper-backed nitrocellulose membrane using a DioDot printing device. The cocktail was imprinted on the cellulose paper in a horizontal line format.

8.4 Preparation of the Detection Test Apparatus

The apparatus 13, illustrated in FIGS. 4 and 5 and comprising a test cartridge 14 and apost-filter cap 15, represents a suitable container to house the test unit 29 and post-filter unit 30 of the present invention.

I. Test Cartridge

The test cartridge, which houses the test unit of the rapid test device, is made of clean technical grade white polypropylene plastic and has a top 16 and bottom 17 component. Both are made in synchronized 16 cavity mold, precisely engineered to allow a snugly fitting tight seal when the two components are pressed together. The components are supplied as individual casings by Top View International Limited, Hong Kong, and are assembled at the manufacturing plant of MedMira Laboratories, Halifax, Canada. These components meet the following criteria:

Appearance—a clear, white smooth texture of the plastic.

A snug fit to produce a leak-proof housing to ensure safe containment of all applied liquids.

Consistency of dimensions to specifications of 2.5 cm width, 3.5 cm length, and 1.3 cm height.

Consistency of dimensions of the reservoir 22 opening of 1.6 cm in diameter and a formed cylinder depth of 0.5 cm.

Consistency in the location of the reservoir 22 in the top component of the test cartridge 14.

A. Reaction Membrane

A porous reaction membrane 21 such as nitrocellulose having an average pore size of 0.45 microns (Whatman, England) is used and cut to 12 mm x 12 mm. The membrane 21 is 0.2 mm paper-backed and specially treated for enhanced protein binding. Certified specifications given by the manufacturer (Whatman, England) include a binding capacity of 80-90 mg protein/cm², a water flow rate of 6 ml/min/cm² and a bubble point of 3.5 bar. The reaction membrane 21 is prepared having two immunoreactive test sites, namely a test zone and a control zone, each zone produced in the shape of a distinct vertical line. The control line and the test line are positioned perpendicular to, but not touching, one another to provide a clear differentiation between the two. The test zone of the membrane Is prepared by applying a solution of vaccinia viral lysate in carbonate buffer (pH 9) using a printer device (BioJet Quanti 3000 dispenser). The control zone is similarly prepared by applying a mixture of a specially calibrated antigen preparation that binds to all classes of IgG antibodies ordinarily present in in a biological fluid sample regardless of HIV IgG antibody status, and thus serves as a control zone. After the membrane is dried at room temperature for 10 minutes, it is treated with a solution of 1% bovine serum albumin in 0.1 M sodium phosphate buffer and allowed to completely dry at ambient temperature for approximately 24 hours.

B. Optional Spacer Layer

A spacer layer 26 supporting the reaction membrane 21 may be produced by securing the outer perimeter of the upper surface of the porous reaction membrane 21 to the lower surface of the porous spacer layer 26 such that the upper surface of the reaction membrane 21 is exposed through the aperture of the spacer layer 26. The upper surface of the reaction membrane 21 is sealed to the lower surface of the spacer layer with a water-insoluble adhesive so as to form an impermeable seal between the rim 28 of the spacer layer 26, defining the aperture 27, and the unexposed upper surface of the reaction membrane 21. This arrangement helps to promote the flow of fluids in a downward, as opposed to lateral, direction through the reaction membrane 21 and into the absorbent pad 20 below. The spacer layer 26 may be purchased with water-soluble adhesive already adhered to the lower surface, or the adhesive may be applied during the manufacturing process. The spacer layer is a polystyrene material insert with a double-sided tape (Halifax Folding Company, Nova Scotia, Canada). The reaction membrane 21 is secured to the spacer layer 26 by the double-sided tape. The assembled spacer layer 26 is approximately 29.0mm×20.5mm in area and 1.0 mm in thickness and is positioned on the upper surface of the absorbent pad 20 which sits in the base of the test cartridge 14 as shown in FIGS. 4 and 5.

C. Absorbent Pad

The absorbent pad 20 is placed directly beneath the reaction membrane 21 and securely inside the bottom member of the test cartridge 14. The pad 20 is composed of thickened compressed cellulose acetate with a porosity of 40 ml/min (Filtrona, Richmond Inc., Richmond, Va.). It is made of synthetic fibers without the use of resins or adhesives and provides an excellent level of aqueous fluid compatibility. Void space is specified at 80 to 85% and absorption of liquids at 6 times the dry unit weight and up to 90% of the total void volume. It is resistant to pH in the range of 2.5 to 9.5. The pad 20 is die cut to a specification of 2.2 cm width, 3.2 cm length and 0.5 cm height. The pad 20 fits securely into the bottom component 17 of the test cartridge 14 so as to create a compressed composite of the reaction membrane 21 with the absorbent pad 20 to ensure a continuum of fluid communication between the porous materials for enhanced hydrodynamics and complete absorption when test samples are applied to the reaction membrane 21.

II. Post-Filter Cap

The post-filter cap 15, which houses the post-filter unit 30 of the rapid test apparatus 13, is comprised of an outer funnel sleeve 32 having an internal collar 37 at its base, an inner funnel sleeve 31 having a handle 38 extending therefrom, and the post-filter unit 30. The funnel sleeves 31,32 and the handle 38 are molded from a plastic material. One preferred plastic material is polystyrene resin (Fouzhou Chimoplus Chemical Company Ltd., China). The outer 32 and inner 31 funnel sleeves are cylindrical in shape with an outside diameter of 15.0 mm and 12.5 mm, respectively and the assembled cap 15 fits snugly into the reservoir 22 of the test cartridge 14. The post-filter cap 15 is designed to be connected to the reservoir 22 of the test cartridge 14 following post-application of the test sample to the reservoir 22, and removed shortly after the push buffer has been added and diffused through the post-filter unit 30 of the filter cap 15. In the assembled form, the volume capacity of the post-filter cap 15 is 0.5 ml.

The post-filter unit 30 is comprised of one filter layer that will be in direct contact with the reaction membrane to improve the subsequent reactivity between the antibodies of the colloidal gold conjugate and the antigen-coated reaction membrane. The filter is comprised of glass micro fiber with OVA binder (Whatman, GF/AVA) which is white and has a basis weight of 48 g/m², a thickness of 303 mm, a flow rate of 150 s/1.5 cm, dry tensile of 640 g/1.5 cm, wet tensile of 324 g/1.5 cm and a porosity of 3 sec/100 ml/in². Once assembled, the freeze-dried colloidal gold conjugate is reconstituted with a solution comprising 0.1-0.15 ml of PBS buffer (0.6-0.7mM potassium chloride, 0.03M sodium chloride, 2-2.1mM di-sodium hydrogen orthophosphate anhydrous, 0.3-0.4 mM potassium phosphate mono) containing 10% sugar. The colloidal gold conjugate solution is dispensed (0.1 to 0.15 ml) onto each filter and then dried at a temperature of 37 to 40° C. The filter layer is die cut according to the following specifications: thickness, 790 to 830 microns; porosity, 1.6 to 2.0 s/100ml/in²; tensile strength 14.5N/55mm; flow rate, 67 s/7.5cm; absorbancy, 76.4%; pore size, 4.3 microns; wicking, 1.00 min:sec; and diameter, 0.42 mm.

8.5 Inspection on Protein A (PA)

Formulation

Sodium Chloride

10% in DDI water

BSA

1% BSA in DDI water pH 5.00 to 9.00 (optimum 6.00).

Colloidal gold

Prepared up to the pH step.

Stock-solution of the Labeling Material

Original concentration diluted in DDI to a final concentration of 0.1- 2 mg/ml (optimum 0.1mg/ml)

Procedure

Prepare a 9 serial dilution Protein A (PA).

To the PA, dilution add the colloidal gold already pH adjusted in a 1:10 ratio (e.g. to 0.1 ml of PA dilution add 1ml of colloidal gold).

Incubate for 10 minutes.

To each dilution tubes add 8-10% of sodium chloride to a final concentration of 1% (optimum 0.9%).

Incubate for 5 minutes.

To each tube again add 0.07- 0.1% of bovine serum albumin to a final concentration of 0.1% (optimum 0.08%).

Read the absorbance at 520 nm.

The correct concentration of protein is the minimal amount that will inhibit flocculation.

| Conc. | Absorbance | Absorbance | |
|---|---|---|---|
| (μg) | 1 | 2 | Average |

| | | | |
|---|---|---|---|
| 0 | 0.313 | 0.314 | 0.314 |
| 2 | 0.524 | 0.524 | 0.524 |
| 3 | 0.533 | 0.532 | 0.533 |
| 4 | 0.533 | 0.532 | 0.533 |
| 5 | 0.540 | 0.540 | 0.540 |
| 6 | 0.575 | 0.571 | 0.573 |
| 7 | 0.580 | 0.580 | 0.580 |
| 8 | 0.576 | 0.583 | 0.580 |
| 9 | 0.576 | 0.576 | 576 |

Hughes D. A. & J. E. Beesley (1998) Preparation of Colloidal Gold Probes In (ed) J. D. Pound. Methods in Molecular Biology vol 80: Immunochemical Protocols, 2^{nd} edition. Humana Press Inc., Totowa, N.J.

8.6 Preparation of Colloidal Gold Conjugate

Materials

| | |
|---|---|
| Sodium Citrate | 0.3 mM in DDI water |
| BSA | 1% BSA in DDI water, pH 5.00 to 9.00 |
| PEG | 1% polyethylene glycol (MW 15,000 to 20,000) in DDI |
| | water, pH to 6.00 |
| Phosphate | mix 0.04 M (in DI water) of NaH₂PO₄ into 0.07 M |
| Buffer | (in water) KH₂PO₄ in a 1:4.4 ratio; pH to 6.00 |
| Protein A and | Protein A and Hepes buffer (0.025 M Hepes and 0.25 |
| Hepes Buffer | mM Thimerosal pH 7.00 in DDI water) in a 1:1 ratio |
| Borate buffer | 0.05 M of sodium borate in DDI water pH 8.50 |

Resuspending Buffer

8mM di-sodium hydrogen orthophosphate anhydrous

1% bovine serum albumin

3mM sodium azide

0.02% polyethylene glycol

0.14- 0.16M sodium chloride

1.5mM potassium dihydrogen orthophosphate

2.7mM potassium chloride.

4.3mM tri-sodium orthophosphate

Mix the above ingredients in 1000 ml of DDI water, pH 7.30 to 7.50.

Procedure

Add 1% of tetrachloroauric acid to water for a final concentration of 0.01%.

Let solution reach a hard boiling point.

Add 15 ml of 0.3mM sodium citrate on a reflux for 30 minutes.

Remove the flask and allow the contents to cool to around 40° C. or lower.

Add 60ml of phosphate buffer (mix 0.04M (In DI water) of NaH₂PO₄ into 0.07M (in water) KH₂PO₄ in a 1:4.4 ratio; pH to 6.000) or 50mM of borate buffer (pH 8.50); adjust pH of the colloidal gold to 6.00- 9.00 (optimal is 6.00), If the pH is too low, add drops of 2mM K₂CO_{3.}

A portion of the solution is removed to perform an aggregation test to know the concentration of the ligand to add to the gold solution.

Add Protein A with a final concentration of 5-9 ug/ml +5% (optimum 6+0.3=6.3 ug/ml)

$$\frac{[ Final PA + 5 \% ] ( mg \text{/} L ) \times total volume in flask ( L )}{[ Initial PA ] ( mg \text{/} ml )}$$

i.e. Total volume=500ml CG and dH2O+15ml sodium citrate+60ml phosphate buffer−3ml test for pH=572ml=0.572l

$$\frac{( 6 + 0.3 ) mg \text{/} l \times 0.572 L}{1 mg \text{/} ml} = 3.60 ml Protein A to be added$$

The solution is allowed to proceed for 15-30 minutes (optimum 20 minutes).

The absorption of the ligand is stop by adding 10% bovine serum albumin pH 5.00 to 9.00 final concentration of 0.1% stir for 5-15 minutes (optimum 10 minutes).

The labeled colloidal gold was centrifuged at a speed of 46,500g (20,000rpm) at a temperature of 4-5°C. for 50 to 80 minutes.

Aspiration and Re-suspension

Aspirate the supernatant with the help of a vacuum flask, taking care not to disturb the pellet. Resuspend in re-suspending buffer (or PBS-BSA) to a final optical density of 1.180 to 4.500 (optimum 2.000) at 520 nm.

Freeze-dry

After the appropriate optical density reach, the solution was filled in 0.6 ml aliquots into a 3 ml glass vials. Some rubber freeze-dry stoppers (1-mm in diameter) were inserted halfway into the vials and transferred into the freeze-dryer shelves. A temperature of −40° C. was maintained for about 5 hours. The primary drying was carried out at a vacuum of less than 100 mTorr with a shelf temperature of −30° C. for about 3 hours and a condenser temperature of less than −80° C. Followed is a shelf temperature of −10° C. for about 5 hours then a shelf temperature of 0° C., vacuum of 0 mTorr for about 2 hours. A secondary drying is carry on at +20° C. for about 4 hours. At the end of the process, the vials were seal under vacuum with the rubber stoppers. The product was then removed from the shelves and a functional test was performed to assure the quality of the product. Samples were kept for later reference.

8.7 Stabilization of Colloidal Gold Conjugate

Procedure

Prepare 1%, 2%, 5% and 10% sucrose in PBS solution, pH 7.0-7.5.

Reconstitute freeze-dried colloidal gold conjugate with a) 5 drops (˜150 μl) b) 10 drops (˜350 μl) and c) 15 drops (˜650 μl) with each percentage of sucrose.

Apply 5 drops (˜150 μl) of each reconstitution to the filter medium.

Let it air dry completely.

Result

| Specimen | 1% | 2% | 5% | 10% |
|---|---|---|---|---|
| Control line | 2+ | 2+ | 2+ | 3+ |
| Positive | 1+ | 1+ | 1+ | 2+/3+ |
| control | | | | |
| Negative | neg | neg | neg | neg |
| control | | | | |

8.8 Preparation of the Post-Filter Unit

One of the goals in diagnostic testing is to develop a test device that requires few manipulative steps. Therefore, by associating the indicator reagent with the filter medium of the post-filter unit 30, it is possible to eliminate extra steps in which the reagents are added separately to the diagnostic device during the detection test protocol.

Materials

Colloidal gold conjugate, Prepared and previously freeze-dried as described above.

Sugar, e.g. trehalose, lactose, sucrose, glucose, maltose, mannose, fructose, etc.

Procedure

Reconstitute the freeze-dried colloidal gold with 0.1- 0.15 ml of PBS buffer (0.6 - 0.7mM potassium chloride, 0.03M Sodium chloride, 2- 2.1mM di-sodium hydrogen orthophosphate anhydrous, 0.3- 0.4 mM potassium phosphate mono) containing 10% sucrose.

Dispense 0.1 to 0.15 ml of colloidal gold solution onto each filter.

Let the filter dry completely at 37- 40° C.

8.9 Preparation of the Push Buffer

Formulation

0.01- 0.1M EDTA

0.02M Sodium azide

0.05- 0.1M Sodium chloride

6mM di-sodium hydrogen orthophosphate anhydrous

0.1- 0.25mM Thimerosal

0.05- 0.1% Triton®X-100

0.02- 0.03M Trizma hydrochloride

0.2- 0.3% Tween-20

0.5- 2.5% PVP-40

Procedure

Add all ingredients together (0.01- 0.1M EDTA, 0.02M sodium azide, 0.05- 0.1M sodium chloride, 6mM di-sodium hydrogen orthophosphate anhydrous, 0.1- 0.25mM Thimerosal, 0.05- 0.1% Triton®X-100, 0.02- 0.03M Trizma hydrochloride, 0.2-0.3% Tween-20, 0.5- 2.5% PVP-40).

Fill up with DDI water.

Adjust the pH to 7.00 to 10.00.

8.10 Detection Test Protocol

Using a clean pipette, 1 drop of a serum or plasma sample was added to the centre of the reaction membrane and the sample allowed to absorb completely through the membrane and into the absorbent material pad. The post-filter cap was connected to the reservoir of the test cartridge so that the post-filter unit was in fluid communication with the test unit. Ten to fifteen drops of the push buffer were subsequently added to the funnel of the post-filter cap. After a brief incubation, about 1 minute, during which time the resolubilized colloidal gold conjugate was drawn through the post filter unit, the post-filter cap was removed from the test cartridge. A distinct colored line(s), one vertical control line and one test line, developed in the centre of the reaction membrane indicating the presence of anti-vaccinia virus antibody in the test sample. The results of the detection test were revealed in about three (3) minutes.

### References

Fenner, F., Henderson, D. A., Arita, I., Jezek, Z., Ladnyi, I. D. Smallpox and Its Eradication. Geneva, Switzerland: World Health Organization (WHO), 1988.

Henderson, D., T. Inglesby, J. Bartlett, M. Ascher E. Eitzen, P. Jahrling, J. Hauer, M. Layton, J. McDade, M. Osterholm, T. O'Toole, G. Parker, T. Perl, P. Russell, & K. Tonat. 1999. Smallpos as a Biological Weapon. JAMA. 281:22. p2127-2137.

Kaufman, J., K. Flanagan, D. Diven, and T. McGovern. 2001. Vaccinia. eMedicine Journal. 2:10.

Roenthal, S., M. Merchlinsky, C. Kleppinger, K. Golenthal. 2001. Developing New Smallpox Vaccines. Emerging Infectious Diseases. 7(6) 920-926.

Shanley, J. 2002. Poxviruses. eMedicine Journal. 3:4.

World Health Organization. 2001. Smallpox. Weekly Epidemiological Record.. 76:44. p. 337-344.

What is claimed is:

1. A device for determining the presence or absence of anti-vaccinia virus antibodies in a fluid test sample, comprising:
a test unit comprising a reaction zone in vertical communication with an absorbent zone, wherein the reaction zone contains a vaccinia viral lysate immobilized therein, said vaccinia viral lysate capable of specific binding with anti-vaccinia virus antibodies present in the fluid test sample to form an immune-complex; and
a post-filter unit comprising a label zone containing a dried indicator reagent, wherein following resolubilization by a buffer reagent, said indicator reagent is capable of specifically binding to the immune-complex to produce a visually detectable signal; and
wherein the reaction zone of the test unit and the label zone of the post-filter unit are capable of being disposed in transient fluid communication with each other so as to allow direct passage of resolubilized indicator reagent from the label zone into the reaction zone following application of the buffer reagent to the label zone.

2. The device according to claim 1, wherein the reaction zone comprises a material which has a pore size permitting separation and filtration of unbound components from the fluid test sample and a thickness which permits an adequate amount of purified vaccinia viral lysate to be immobilized thereto.

3. The device according to claim 2, wherein the pore size of the material ranges from about 0.1 to 12.0 microns and the thickness of the material ranges from about 0.05 mm to 3.0 mm.

4. The device according to claim 2, wherein non-specific binding sites on a surface of the material are inactivated by application of a protein blocking agent.

5. The device according to claim 2, wherein the material is a nitrocellulose membrane backed with porous paper.

6. The device according to claim 1, wherein the vaccinia viral lysate is immobilized within a discrete test zone of the reaction zone by absorption using a dispenser/printer technique.

7. The device according to claim 1, wherein the reaction zone further comprises an immobilized control reagent in a discernable and separate area from the vaccinia viral lysate, said control reagent comprising an antibody against a human immunoglobulin selected from the group consisting of IgG, IgM, IgE and IgA.

8. The device according to claim 1, wherein the absorbent zone is separated from the reaction zone by an intervening spacer layer having one or more openings defined therein to permit fluid communication between the reaction zone and the absorbent zone.

9. The device according to claim 1, wherein the absorbent zone comprises one or more layers of a wicking material which is capable of wicking fluid by capillary action and absorbing a substantial volume of fluid.

10. The device according to claim 9, wherein the wicking material is cellulose acetate.

11. The device according to claim 1, wherein the label zone comprises a filter material having a pore size capable of allowing the dried indicator reagent to be effectively resolubilized by the buffer reagent and transferred to the reaction zone by laminar fluid flow.

12. The device according to claim 11, wherein the filter material is glass fiber filter paper.

13. The device according to claim 1, wherein the indicator reagent comprises a direct label.

14. The device according to claim 13, wherein the indicator reagent is a colloidal gold-Protein A conjugate.

15. The device according to claim 1, wherein the post filter unit is pretreated with a glazing material in the region to which the indicator reagent is applied.

16. The device according to claim 1, wherein said test unit and said post-filter unit are contained in a housing.

17. The device according to claim 1, further comprising a blood separation zone in lateral communication with the reaction zone, wherein the blood separation zone comprises:
a first end defining a region for receiving a whole blood test sample, and
a second end in direct communication with the reaction zone,
wherein the blood separation zone comprises a material capable of selectively retaining red blood cells from a whole blood test sample to generate a substantially red blood cell-free fluid portion, and allowing flow of said fluid portion from the first end of the blood separation zone to the reaction zone.

18. The device according to claim 17, wherein the material capable of selectively retaining red blood cells is a glass fiber material having dimensions between about 4 and 7 mm in width, between about 10 and 15 mm in length and between about 0.2 mm and 1.0 mm in thickness.

19. The device according to claim 17, wherein the material capable of selectively retaining red blood cells comprises a hydrophobic carrier or backing capable of reducing seepage of the whole blood test sample and the red blood cell-free fluid portion as it migrates along the blood separation zone.

20. The device according to claim 19, wherein the hydrophobic carrier or backing is comprised of a material selected from the group consisting of polycarbonate, polyethylene, MylarTM, polypropylene, vinyl, cellophane, polystyrene, water-proofed cardboard and water-resistant cardboard.

21. A method for determining the presence or absence of anti-vaccinia virus antibodies in a fluid test sample, comprising the steps of:
depositing the fluid test sample onto the reaction zone of the test unit of a device as defined in claim 1;
bringing the label zone of the post-filter unit and the reaction zone of the test unit into fluid communication therewith;
applying a buffer reagent to the post-filter unit to reconstitute the dried indicator reagent and wash any unbound reactants from the reaction zone; and
removing the post-filter unit to observe a test result depicted by a presence or absence of a visually detectable signal on the reaction zone.

22. A method for determining the presence or absence of anti-vaccinia virus antibodies in a whole blood test sample, comprising the steps of:
depositing the whole blood test sample onto the first end of the blood separation zone of a device as defined in claim 17;
bringing the label zone of the post-filter unit and the reaction zone of the test unit into fluid communication therewith;
applying a buffer reagent to the post-filter unit to reconstitute the dried indicator reagent and wash any unbound reactants from the reaction zone; and
removing the post-filter unit to observe a test result depicted by a presence or absence of a visually detectable signal on the reaction zone.
